(19) United States
(12) Patent Application Publication
(10) Pub. No.: US 2023/0381271 A1
(43) Pub. Date: Nov. 30, 2023

(54) PROTOCOL TO MINIMIZE CALCINEURIN INHIBITOR NEPHROTOXICITY

(71) Applicant: Aurinia Pharmaceuticals Inc., Edmonton (CA)

(72) Inventors: Michael Martin, Victoria (CA); Robert B. Huizinga, Victoria (CA); Neil Solomons, Victoria (CA)

(73) Assignee: Aurinia Pharmaceuticals Inc., Edmonton (CA)

(21) Appl. No.: 18/361,775

(22) Filed: Jul. 28, 2023

Related U.S. Application Data
(63) Continuation of application No. 18/272,337, filed on Jan. 1, 0001.
(60) Provisional application No. 63/246,765, filed on Sep. 21, 2021.
(60) Provisional application No. 63/245,779, filed on Sep. 17, 2021.
(60) Provisional application No. 63/138,325, filed on Jan. 15, 2021.

(51) Int. Cl. A61K 38/13; A61K 31/5377; A61K 31/573; A61P 37/06; A61P 13/12; G01N 33/70; G01N 33/62; G01N 33/68; G01N 33/92; G01N 33/53

(52) U.S. Cl. CPC A61K 38/13; A61K 31/5377; A61K 31/573; A61P 37/06; A61P 13/12; G01N 33/70; G01N 33/62; G01N 33/6893; G01N 33/92; G01N 33/5308; G01N 2800/52; G01N 2800/347

(57) ABSTRACT

Provided herein are methods of employing pharmacodynamics regimens to maximize effectiveness of voclosporin in treatment of proteinuric kidney diseases while minimizing undesirable side effects, such as but not limited to calcineurin inhibitor nephrotoxicity. Also provided are methods of assessments of renal functions and/or conditions, and corresponding protocols to modify, stop, restore and/or re-initiate voclosporin dosing and administrations to maximize effectiveness of voclosporin in treatment of proteinuric kidney diseases while minimizing undesirable side effects.

### CROSS REFERENCE TO RELATED APPLICATIONS

The present application is a continuation of U.S. patent application Ser. No. 18/272,337, filed Jul. 13, 2023, which claims priority to national stage application under 35 U.S.C. § 371 of International Application No. PCT/IB2022/050316, filed internationally on Jan. 14, 2022, which claims priority from U.S. Provisional Patent Application No. 63/138,325, filed Jan. 15, 2021, entitled “PROTOCOL TO MINIMIZE CALCINEURIN INHIBITOR NEPHROTOXICITY,” U.S. Provisional Patent Application No. 63/245,779, filed Sep. 17, 2021, entitled “PROTOCOL TO MINIMIZE CALCINEURIN INHIBITOR NEPHROTOXICITY,” and U.S. Provisional Patent Application No. 63/246,765, filed Sep. 21, 2021, entitled “PROTOCOL TO MINIMIZE CALCINEURIN INHIBITOR NEPHROTOXICITY,” the contents of which are incorporated by reference in their entirety.

### FIELD

The present disclosure relates to methods of employing pharmacodynamics regimens to maximize effectiveness of voclosporin in treatment of proteinuric kidney diseases while minimizing undesirable side effects, such as but not limited to calcineurin inhibitor nephrotoxicity. Also provided are methods of assessments of renal functions and/or conditions, and corresponding protocols to modify, stop, restore and/or re-initiate voclosporin dosing and administrations to maximize effectiveness of voclosporin in treatment of proteinuric kidney diseases while minimizing undesirable side effects.

### BACKGROUND

Calcineurin inhibitor drugs (CNI), such as cyclosporine and tacrolimus, both potent immunosuppressant, and have been tested for use in the treatment of a variety of conditions, including use in conjunction with tissue transplant. Both of these compounds show long term nephrotoxicity.

It would be beneficial to develop a CNI that was an effective immunosuppressant that is also tolerated for long term use. In particular, it would be beneficial to identify such a CNI and a method of administering same for prolonged periods of time.

### SUMMARY

Provided herein in some aspects are methods, compositions, devices, and systems for reducing chronic calcineurin inhibitor nephrotoxicity in the treatment of proteinuric kidney disease or associated with a transplant, including identifying subjects as appropriate for said method, administering a predetermined daily dosage of effective amounts of voclosporin, and evaluating said subjects for renal function at a time point before, during and after the end of said treatment period.

In some of any embodiments, the method to reduce chronic calcineurin inhibitor nephrotoxicity in treatment of a proteinuric kidney disease or associated with a transplant disclosed herein involves administering to a subject diagnosed with said disease or a subject that is receiving or is a candidate for receiving a transplant a predetermined daily dosage of effective amounts of voclosporin over a projected treatment period of at least 55 weeks. In some examples, the method further includes assessing the estimated Glomerular Filtration Rate (eGFR) of the subject at at least a first time point and a second time point on different days of said treatment period, and if the eGFR of said subject decreases by more than a target % to below a predetermined value, between said first and second time points, reducing the daily dosage by increment(s) of 7.9 mg BID or stopping the administering of voclosporin to said subject. In some examples, the method further includes assessing the estimated Glomerular Filtration Rate (eGFR) of the subject at at least a first time point and a second time point on different days of said treatment period, and if the eGFR of the subject decreases by less than said target %, between said first and second time points, the method includes continuing administering the same predetermined daily dosage of voclosporin to said subject.

In some of any embodiments, the first time point is immediately preceding initiating said protocol. In some examples, the predetermined value is in the range of 50-90 ml/min/1.73 m². In some of any embodiments, the target % is in the range of 20-45%. In some aspects, the predetermined value is approximately 60 ml/min/1.73 m². In some examples, the target % is approximately 30%.

In some of any embodiments, the method disclosed herein further includes identifying a subject as appropriate for said method prior to conducting said method on the subject by (a) determining that the urine protein creatinine ratio (UPCR) of the subject is >1 mg/mg as measured by first morning void or 24 hour urine, and (b) determining that the subject has an eGFR as measured by Chronic Kidney Disease Epidemiology Collaboration equation (CKD-EP1) of >45 ml/min/1.73 m²; if the conditions of (a) and (b) are met, the subject is identified as appropriate for said method.

In some of any embodiments, the method to reduce chronic calcineurin inhibitor nephrotoxicity in treatment of a proteinuric kidney disease or associated with a transplant disclosed herein involves administering to a subject diagnosed with said disease, a subject that is receiving or is a candidate for receiving a transplant, predetermined daily dosages of effective amounts of voclosporin over a projected treatment period to an end point, further comprising: (a) measuring urinary protein creatinine ratio (UPCR) of said subject at a first time point prior to said treatment period and a second time point occurring prior to the end point but after the start of the treatment period and determining any reduction of said UPCR, between said first and second time points, and (b) if the UPCR of said subject fails to show a reduction of at least a predetermined amount at said second time point, discontinuing administering voclosporin to the subject and continuing said administering if said predetermined amount of reduction is shown.

In some of any embodiments, the method described herein further comprises measuring the concentration of C3/C4 in the blood of said subject at said first and second time points and determining whether the concentration of C3/C4 is normalized at said second time point and if said normalization is found, reinstating or continuing administering voclosporin to the subject and if normalization has not occurred maintaining said discontinuing.

In some of any embodiments, the method to treat a proteinuric kidney disease disclosed herein involves administering to a subject diagnosed with the proteinuric kidney disease, a predetermined daily dosage of effective amounts of voclosporin over a projected treatment period of at least 8 weeks, wherein said effective amount is 15.8 mg BID or 7.9 mg voclosporin BID.

In some of any embodiments, the method to reduce chronic calcineurin inhibitor nephrotoxicity associated with a transplant disclosed herein involves administering to a subject that is receiving or is a candidate for receiving a transplant, a predetermined daily dosage of effective amounts of voclosporin over a projected treatment period of at least 8 weeks, wherein said effective amount is 15.8 mg BID or 7.9 mg voclosporin BID.

In some of any embodiments, the method to reduce transplant rejection disclosed herein involves administering to a subject that is receiving or is a candidate for receiving a transplant, a predetermined daily dosage of effective amounts of voclosporin over a projected treatment period of at least 8 weeks, wherein said effective amount is 15.8 mg BID or 7.9 mg voclosporin BID.

In some of any embodiments, the proteinuric kidney disease is lupus nephritis.

In some of any embodiments, the transplant is an organ transplant or a tissue transplant. In some of any embodiments, the organ transplant is a kidney (renal) transplant, a liver transplant, or a heart transplant. In some of any embodiments, the organ transplant is a kidney (renal) transplant.

In some of any embodiments, the subject has increased susceptibility to chronic calcineurin inhibitor nephrotoxicity. In some aspects, the subject has increased susceptibility to chronic calcineurin inhibitor nephrotoxicity and exhibits one or more of (a) variability in P-glycoprotein expression and/or activity, (b) variability in CYP3A4/5 expression and activity, (c) older kidney age, (d) salt depletion, (e) the use of nonsteroidal anti-inflammatory drugs, (f) and has genetic polymorphisms in TGF-β and/or ACE. In some aspects, the subject has increased susceptibility to chronic calcineurin inhibitor nephrotoxicity and exhibits one or more of (a) variability in P-glycoprotein expression and/or activity, (b) variability in CYP3A4/5 expression or activity, (c) older kidney age, (d) salt depletion, (e) the use of nonsteroidal anti-inflammatory drugs, (f) or has genetic polymorphisms in TGF-β and/or ACE.

In some of any embodiments, the method disclosed herein further includes assessing the interstitial fibrosis and tubular atrophy of said subject by renal biopsies at at least a first time point and a second time point on different days of said treatment period, and if interstitial fibrosis and tubular atrophy is observed in >5% in cortical area, between said first and second time points, reducing the daily dosage by increment(s) of 7.9 mg BID or stopping the administering of voclosporin to said subject. In some of any embodiments, the method disclosed herein further includes assessing the interstitial fibrosis and tubular atrophy of said subject by renal biopsies at at least a first time point and a second time point on different days of said treatment period, and if interstitial fibrosis and tubular atrophy is observed in <5% in cortical area, between said first and second time points, continuing administering the same predetermined daily dosage of voclosporin to said subject.

In some of any embodiments, the method disclosed herein further includes assessing the presence of medial arteriolar hyalinosis of said subject by renal biopsies at at least a first time point and a second time point on different days of said treatment period, and if medial arteriolar hyalinosis is present, between said first and second time points, reducing the daily dosage by increment(s) of 7.9 mg BID or stopping the administering of voclosporin to said subject. In some of any embodiments, the method disclosed herein further includes assessing the presence of medial arteriolar hyalinosis of said subject by renal biopsies at at least a first time point and a second time point on different days of said treatment period, and if medial arteriolar hyalinosis is not present, between said first and second time points, continuing administering the same predetermined daily dosage of voclosporin to said subject. In some aspects, medial arteriolar hyalinosis is identified by the replacement of necrotic smooth muscle cells with focal, circular lumpy protein (hyaline) deposits at the periphery of the wall of afferent arterioles, and the narrowing of the vascular lumen. In some aspects, the medial arteriolar hyalinosis is identified by the replacement of necrotic smooth muscle cells with focal, circular lumpy protein (hyaline) deposits at the periphery of the wall of afferent arterioles, or the narrowing of the vascular lumen.

In some of any embodiments, the method disclosed herein further includes assessing the presence of glomerular injury of said subject by renal biopsies at at least a first time point and a second time point on different days of said treatment period, and if glomerular injury is present, between said first and second time points, reducing the daily dosage by increment(s) of 7.9 mg BID or stopping the administering of voclosporin to said subject. In some of any embodiments, the method disclosed herein further includes assessing the presence of glomerular injury of said subject by renal biopsies at at least a first time point and a second time point on different days of said treatment period, and if glomerular injury is not present, between said first and second time points, continuing administering the same predetermined daily dosage of voclosporin to said subject. In some aspects, the glomerular injury comprises global and segmental glomerulosclerosis, tubular atrophy, interstitial fibrosis, and arteriosclerosis. In some aspects, the glomerular injury comprises global and segmental glomerulosclerosis, tubular atrophy, interstitial fibrosis, or arteriosclerosis. In some examples, the glomerular injury is present when total renal chronicity score is >1.

In some of any embodiments, the method disclosed herein further includes assessing the presence of juxtaglomerular apparatus (JGA) hyperplasia of said subject by renal biopsies at at least a first time point and a second time point on different days of said treatment period, and if JGA hyperplasia is present, between said first and second time points, reducing the daily dosage by increment(s) of 7.9 mg BID or stopping the administering of voclosporin to said subject. In some of any embodiments, the method disclosed herein further includes assessing the presence of juxtaglomerular apparatus (JGA) hyperplasia of said subject by renal biopsies at at least a first time point and a second time point on different days of said treatment period, and if JGA hyperplasia is not present, between said first and second time points, continuing administering the same predetermined daily dosage of voclosporin to said subject. In some examples, JGA hyperplasia includes enlargements of juxtaglomerular apparatus components comprising one or more of: the vascular components, the mesangial cell components, the tubular components (the macula densa), and the presence of intracellular renin granules. In some examples, the JGA hyperplasia includes enlargements of juxtaglomerular apparatus components comprising one or more of: the vascular components, the mesangial cell components, the tubular components (the macula densa), or the presence of intracellular renin granules.

In some of any embodiments, the method disclosed herein further includes assessing the presence of tubular microcalcifications of said subject by renal biopsies at at least a first time point and a second time point on different days of said treatment period, and if tubular microcalcifications is present, between said first and second time points, reducing the daily dosage by increment(s) of 7.9 mg BID or stopping the administering of voclosporin to said subject. In some of any embodiments, the method disclosed herein further includes assessing the presence of tubular microcalcifications of said subject by renal biopsies at at least a first time point and a second time point on different days of said treatment period, and if tubular microcalcifications is not present, between said first and second time points, continuing administering the same predetermined daily dosage of voclosporin to said subject.

In some of any embodiments, the method disclosed herein further includes assessing the P-glycoprotein (P-gp) expression of said subject by renal biopsies at at least a first time point and a second time point on different days of said treatment period, and if loss of expression of P-gp is more than a predetermined value, between said first and second time points, reducing the daily dosage by increment(s) of 7.9 mg BID or stopping the administering of voclosporin to said subject. In some of any embodiments, the method disclosed herein further includes assessing the P-glycoprotein (P-gp) expression of said subject by renal biopsies at at least a first time point and a second time point on different days of said treatment period, and if loss of expression of P-gp is less than the predetermined value, between said first and second time points, continuing administering the same predetermined daily dosage of voclosporin to said subject. In some examples, the predetermined value for loss of expression of P-gp is 10% loss of P-gp expression in tubules in the cortical area.

In some of any embodiments, the method disclosed herein further includes assessing the Calcineurin Inhibitor (CNI) Nephrotoxicity and/or Banff Scores of said subject by renal biopsies at at least a first time point and a second time point on different days of said treatment period, and if the CNI Nephrotoxicity and/or Banff Scores are outside of predetermined ranges, between said first and second time points, reducing the daily dosage by increment(s) of 7.9 mg BID or stopping the administering of voclosporin to said subject. In some of any embodiments, the method disclosed herein further includes assessing the Calcineurin Inhibitor (CNI) Nephrotoxicity and/or Banff Scores of said subject by renal biopsies at at least a first time point and a second time point on different days of said treatment period, and if the CNI Nephrotoxicity and/or Banff Scores are within predetermined ranges, between said first and second time points, continuing administering the same predetermined daily dosage of voclosporin to said subject. In some aspects, the predetermined range of CNI Nephrotoxicity Score is 0-3, and the predetermined range of Banff Score is 0-3. In some aspects, the predetermined range of CNI Nephrotoxicity Score is 0-3, or the predetermined range of Banff Score is 0-3.

In some of any embodiments, the method disclosed herein further includes assessing one or more of the National Institutes of Health Activity Index (NIH-AI), the National Institutes of Health Chronicity Index (NIH-CI), and the Tubulointerstitial Activity Index (TIAI) of said subject by renal biopsies at at least a first time point and a second time point on different days of said treatment period, and if the NIH-AI, NIH-CI, and/or TIAI are outside of predetermined ranges, between said first and second time points, reducing the daily dosage by increment(s) of 7.9 mg BID or stopping the administering of voclosporin to said subject. In some of any embodiments, the method disclosed herein further includes assessing one or more of the National Institutes of Health Activity Index (NIH-AI), the National Institutes of Health Chronicity Index (NIH-CI), and the Tubulointerstitial Activity Index (TIAI) of said subject by renal biopsies at at least a first time point and a second time point on different days of said treatment period, and if the NIH-AI, NIH-CI, and/or TIAI are within predetermined ranges, between said first and second time points, continuing administering the same predetermined daily dosage of voclosporin to said subject. In some of any embodiments, the predetermined range of NIH-AI is 0-6, and the predetermined range of NIH-CI is 0-3, and the predetermined range of TIAI is 0-5. In some of any embodiments, the predetermined range of NIH-AI is 0-6, or the predetermined range of NIH-CI is 0-3, and the predetermined range of TIAI is 0-5. In some of any embodiments, the predetermined range of NIH-AI is 0-6, and the predetermined range of NIH-CI is 0-3, or the predetermined range of TIAI is 0-5. In some of any embodiments, the predetermined range of NIH-AI is 0-6, or the predetermined range of NIH-CI is 0-3, or the predetermined range of TIAI is 0-5.

In some of any embodiments, the method disclosed herein further includes assessing urine anion gap (UAG) in urinary specimens of said subject at at least a first time point and a second time point on different days of said treatment period, and if the UAG is outside of a predetermined range, between said first and second time points, reducing the daily dosage by increment(s) of 7.9 mg BID or stopping the administering of voclosporin to said subject. In some of any embodiments, the method disclosed herein further includes assessing urine anion gap (UAG) in urinary specimens of said subject at at least a first time point and a second time point on different days of said treatment period, and if the UAG is within the predetermined range, between said first and second time points, continuing administering the same predetermined daily dosage of voclosporin to said subject. In some aspects, the predetermined range of UAG is between 20-90 mEq/L.

In some of any embodiments, the method disclosed herein further includes assessing hyperkalemia, hypomagnesemia, magnesium wasting and hyperuricemia in serum and urine samples of said subject at at least a first time point and a second time point on different days of said treatment period, and if hyperkalemia, hypomagnesemia, magnesium wasting and hyperuricemia are detected, between said first and second time points, reducing the daily dosage by increment(s) of 7.9 mg BID or stopping the administering of voclosporin to said subject. In some of any embodiments, the method disclosed herein further includes assessing hyperkalemia, hypomagnesemia, magnesium wasting and/or hyperuricemia in serum and urine samples of said subject at at least a first time point and a second time point on different days of said treatment period, and if hyperkalemia, hypomagnesemia, magnesium wasting or hyperuricemia are detected, between said first and second time points, reducing the daily dosage by increment(s) of 7.9 mg BID or stopping the administering of voclosporin to said subject. In some of any embodiments, the method disclosed herein further includes assessing hyperkalemia, hypomagnesemia, magnesium wasting and hyperuricemia in serum and urine samples of said subject at at least a first time point and a second time point on different days of said treatment period, and if hyperkalemia, hypomagnesemia, magnesium wasting and hyperuricemia are not detected, between said first and second time points, continuing administering the same predetermined daily dosage of voclosporin to said subject. In some of any embodiments, the method disclosed herein further includes assessing hyperkalemia, hypomagnesemia, magnesium wasting and hyperuricemia in serum and urine samples of said subject at at least a first time point and a second time point on different days of said treatment period, and if hyperkalemia, hypomagnesemia, magnesium wasting or hyperuricemia are not detected, between said first and second time points, continuing administering the same predetermined daily dosage of voclosporin to said subject. In some aspects, hyperkalemia is determined by a serum potassium level of >5 mmol/L. In some aspects, hypomagnesemia is determined by a serum magnesium level less than 1.4 mg/dL. In some aspects, magnesium wasting is determined by a urine magnesium level of more than 2 mEq. In some aspects, hyperuricemia is determined by a serum uric acid level of >7.0 mg/dL

In some of any embodiments, the method disclosed herein further includes assessing serum creatinine (SCr) and serum cystatin C (SCysC) in serum samples of said subject at at least a first time point and a second time point on different days of said treatment period, and if the SCr and SCysC levels are elevated above predetermined ranges, between said first and second time points, reducing the daily dosage by increment(s) of 7.9 mg BID or stopping the administering of voclosporin to said subject. In some of any embodiments, the method disclosed herein further includes assessing serum creatinine (SCr) and serum cystatin C (SCysC) in serum samples of said subject at at least a first time point and a second time point on different days of said treatment period, and if the SCr or SCysC levels are elevated above predetermined ranges, between said first and second time points, reducing the daily dosage by increment(s) of 7.9 mg BID or stopping the administering of voclosporin to said subject. In some of any embodiments, the method disclosed herein further includes assessing serum creatinine (SCr) and serum cystatin C (SCysC) in serum samples of said subject at at least a first time point and a second time point on different days of said treatment period, and if the SCr and SCysC levels are within predetermined ranges, between said first and second time points, continuing administering the same predetermined daily dosage of voclosporin to said subject. In some of any embodiments, the method disclosed herein further includes assessing serum creatinine (SCr) and serum cystatin C (SCysC) in serum samples of said subject at at least a first time point and a second time point on different days of said treatment period, and if the SCr or SCysC levels are within predetermined ranges, between said first and second time points, continuing administering the same predetermined daily dosage of voclosporin to said subject. In some examples, the predetermined range of SCr level is 0.84-1.21 mg/dL, and the predetermined range of SCysC level is below 1 mg/L. In some examples, the predetermined range of SCr level is 0.84-1.21 mg/dL, or the predetermined range of SCysC level is below 1 mg/L.

In some of any embodiments, the method disclosed herein further includes assessing creatinine clearance (CrCl) and blood urea nitrogen (BUN) in serum samples of said subject at at least a first time point and a second time point on different days of said treatment period, and if the CrCl level is decreased below a first predetermined range and if BUN level is elevated above a second predetermined range, between said first and second time points, reducing the daily dosage by increment(s) of 7.9 mg BID or stopping the administering of voclosporin to said subject. In some of any embodiments, the method disclosed herein further includes assessing creatinine clearance (CrCl) and blood urea nitrogen (BUN) in serum samples of said subject at at least a first time point and a second time point on different days of said treatment period, and if the CrCl level is decreased below a first predetermined range or if BUN level is elevated above a second predetermined range, between said first and second time points, reducing the daily dosage by increment(s) of 7.9 mg BID or stopping the administering of voclosporin to said subject. In some of any embodiments, the method disclosed herein further includes assessing creatinine clearance (CrCl) and blood urea nitrogen (BUN) in serum samples of said subject at at least a first time point and a second time point on different days of said treatment period, and if the CrCl and BUN levels are within predetermined ranges, between said first and second time points, continuing administering the same predetermined daily dosage of voclosporin to said subject. In some of any embodiments, the method disclosed herein further includes assessing creatinine clearance (CrCl) and blood urea nitrogen (BUN) in serum samples of said subject at at least a first time point and a second time point on different days of said treatment period, and if the CrCl or BUN levels are within predetermined ranges, between said first and second time points, continuing administering the same predetermined daily dosage of voclosporin to said subject. In some aspects, the first predetermined range of CrCl level is 137-150 mL/min in males and 128-130 mL/min in females, and the second predetermined range of BUN level is 7 to 20 mg/dL.

In some of any embodiments, the method disclosed herein further includes assessing renal vascular resistance and renal plasma flow (RPF) of said subject at at least a first time point and a second time point on different days of said treatment period, and if the renal vascular resistance and RPF are altered outside of predetermined values, between said first and second time points, reducing the daily dosage by increment(s) of 7.9 mg BID or stopping the administering of voclosporin to said subject. In some of any embodiments, the method disclosed herein further includes assessing renal vascular resistance and renal plasma flow (RPF) of said subject at at least a first time point and a second time point on different days of said treatment period, and if the renal vascular resistance or RPF are altered outside of predetermined values, between said first and second time points, reducing the daily dosage by increment(s) of 7.9 mg BID or stopping the administering of voclosporin to said subject. In some of any embodiments, the method disclosed herein further includes assessing renal vascular resistance and renal plasma flow (RPF) of said subject at at least a first time point and a second time point on different days of said treatment period, and if the renal vascular resistance and RPF remain within predetermined values, between said first and second time points, continuing administering the same predetermined daily dosage of voclosporin to said subject. In some of any embodiments, the method disclosed herein further includes assessing renal vascular resistance and renal plasma flow (RPF) of said subject at at least a first time point and a second time point on different days of said treatment period, and if the renal vascular resistance or RPF remain within predetermined values, between said first and second time points, continuing administering the same predetermined daily dosage of voclosporin to said subject. In some examples, the predetermined value of RPF is 600 mL/min.

In some of any embodiments, the method disclosed herein further includes assessing albuminuria in morning and random urinary specimens of said subject at at least a first time point and a second time point on different days of said treatment period, and if the albuminuria is detected, between said first and second time points, reducing the daily dosage by increment(s) of 7.9 mg BID or stopping the administering of voclosporin to said subject. In some of any embodiments, the method disclosed herein further includes assessing albuminuria in morning and random urinary specimens of said subject at at least a first time point and a second time point on different days of said treatment period, and if the albuminuria is not detected, between said first and second time points, continuing administering the same predetermined daily dosage of voclosporin to said subject. In some aspects, albuminuria is determined by the presence of albumin/creatinine ratios of >30 mg/g.

In some of any embodiments, the methods described herein do not substantially decrease or increase urinary electrolyte levels, between said first and second time points. In some of any embodiments, the method does not result in a substantial increase or decrease of the level of one or more urinary electrolytes, or the level of one or more urinary electrolytes is decreased or increased by less than the predetermined value, between said first and second time points.

In some of any embodiments, provided herein is a method involving (a) assessing the level of one or more urinary electrolytes of said subject at at least a first time point and a second time point on different days of said treatment period, and (b) (i) if the level of the one or more urinary electrolyte is decreased or increased by more than a predetermined value, between said first and second time points, reducing the daily dosage by increment(s) of 7.9 mg BID or stopping the administering of voclosporin to said subject; (ii) if the level of the one or more urinary electrolyte is decreased or increased by less than the predetermined value, between said first and second time points, continuing administering the same predetermined daily dosage of voclosporin to said subject.

In some of any embodiments, the urinary electrolyte is selected from one or more of magnesium, sodium and potassium. In some of any embodiments, the urinary electrolyte is magnesium, and the predetermined value is about 20 mg/dL. In some of any embodiments, the urinary electrolyte is sodium, and the predetermined value is about 50 mmol/L. In some of any embodiments, the urinary electrolyte is potassium, and the predetermined value is about 10 mmol/L.

In some of any embodiments, the methods described herein do not result in a substantial dyslipidemia, or the level of one or more lipids is within a predetermined range, at said second time point.

In some of any embodiments, the method disclosed herein further includes (a) assessing the level of one or more lipids of said subject at at least a first time point and a second time point on different days of said treatment period, and (b) (i) if the level of the one or more lipid is outside of a predetermined range, at said second time point, reducing the daily dosage by increment(s) of 7.9 mg BID or stopping the administering of voclosporin to said subject; (ii) if the level of the one or more lipid is within a predetermined range, at said second time point, continuing administering the same predetermined daily dosage of voclosporin to said subject. In some of any embodiments, the one or more lipid is selected from one or more of total cholesterol, low density lipoprotein (LDL) cholesterol, and triglyceride. In some of any embodiments, the lipid is total cholesterol, and the predetermined range is 100-200 mg/dL. In some of any embodiments, the lipid is triglycerides, and the predetermined range is 50-150 mg/dL. In some of any embodiments, the lipid is LDL, and the predetermined range is 50-130 mg/dL.

In any of the provided embodiments, the first time point is immediately provided initiating said protocol. In some of any embodiments, the second time point is after the first time point and initiating said protocol. In some of any embodiments, the second time point is after the mid-point of the projected treatment period. In some of any embodiments, the second time point is after the end of the projected treatment period.

In any of the provided embodiments, the predetermined daily dosage is 39.5 mg voclosporin BID or 31.6 mg voclosporin BID or 23.7 mg voclosporin BID or 15.8 mg voclosporin BID or 7.9 mg voclosporin BID.

In any of the provided embodiments, the method further includes evaluating said subject for renal function at a time point after the end of said treatment period by assessing eGFR. In some of any embodiments, the method further includes evaluating said subject for efficacy by assessing protein/creatinine ratio (UPCR) at a time point after the end of said treatment period.

In any of the provided embodiments, the method further includes administering to said subject an effective amount of mycophenolate mofetil (MMF). In some of any embodiments, the method further includes administering to said subject an effective amount of a corticosteroid. In some of any embodiments, the treatment period is at least 100 weeks. In some of any embodiments, the treatment period is at least 150 weeks.

In any of the provided embodiments, the method further includes determining the eGFR of the subject at a third time point and if the eGFR is determined at said third time point to differ from the eGFR determined at said first time point by less than said target %, resuming administering said predetermined daily dosage of voclosporin. In some examples, the target % is 20-45%. In some examples, the target % is approximately 30%.

In some of any embodiments, disclosed herein is a method to reduce chronic calcineurin inhibitor nephrotoxicity in treatment of a proteinuric kidney disease, in which the method includes administering to a subject diagnosed with said disease predetermined daily dosages of effective amounts of voclosporin over a projected treatment period to an end point, and further includes measuring urinary protein creatinine ratio (UPCR) of said subject at a first time point prior to said treatment period and a second time point occurring prior to the end point but after the start of the treatment period and determining any reduction of said UPCR, between said first and second time points, and, if the UPCR of said subject fails to show a reduction of at least a predetermined amount at said second time point, discontinuing administering voclosporin to the subject and continuing said administering if said predetermined amount of reduction is shown.

In some of any embodiments, the method described herein further comprises measuring the concentration of C3/C4 in the blood of said subject at said first and second time points and determining whether the concentration of C3/C4 is normalized at said second time point and, if said normalization is found, reinstating or continuing administering voclosporin to the subject and if normalization has not occurred maintaining said discontinuing. In some aspects, the method further includes administering to said subject an effective amount of mycophenolate mofetil (MMF). In some examples, the method further includes administering to the subject an effective amount of a corticosteroid. In some of any embodiments, the predetermined daily dosage is 39.5 mg voclosporin BID or 31.6 mg voclosporin BID or 23.7 mg voclosporin BID or 15.8 mg voclosporin BID or 7.9 mg voclosporin BID.

In some of any embodiments, disclosed herein is a method to treat a proteinuric kidney disease which includes administering to a subject diagnosed with lupus nephritis a predetermined daily dosage of effective amounts of voclosporin over a projected treatment period of at least 8 weeks, wherein said effective amount is 15.8 mg BID or 7.9 mg voclosporin BID.

### BRIEF DESCRIPTION OF THE DRAWINGS

FIG. 1 shows a schematic representation of a trial comparing the safety and efficacy of voclosporin to placebo at 24 and 48 weeks in subjects concurrently administered mycophenolate mofetil (MMF) and oral corticosteroids (top panel), which were subject to the presented tapering protocol (bottom panel).

FIG. 2A-FIG. 2K shows the structure of various voclosporin metabolites.

FIG. 2A shows voclosporin metabolite IM4n. FIG. 2B shows voclosporin metabolite IM9, the primary metabolite. FIG. 2C shows voclosporin metabolite IM1c (R). FIG. 2D shows voclosporin metabolite IM1c (S). FIG. 2E shows voclosporin metabolite IM19 (MeBmt Cyclization). FIG. 2F shows voclosporin metabolite IM19. FIG. 2G shows voclosporin metabolite IM4. FIG. 2H shows voclosporin metabolite IM1-Diol-1. FIG. 2I shows voclosporin metabolite IM1-Diol-2. FIG. 2J shows voclosporin metabolite IMI-Diol-3, a diastereomer of IMI-Diol-2 differing in configuration at the 1−η carbon of the aa-1. FIG. 2K shows a hypothetical voclosporin metabolite with trihydroxylation.

FIG. 3 shows MALDI-MSI imaging of cyclosporine A (CsA), voclosporin (VSP), and their metabolites in a mouse kidney after IV administration of either parent drug. Mass to signal ratio is visualized as color intensity maps (m/z intensity) to provide spatial information for the detected molecules.

FIG. 4A-FIG. 4E shows a series of overlaid and separate in situ MALDI-MSI images for cyclosporine A (CsA) or voclosporin (VSP) parent drug and their metabolites in a mouse kidney following administration. FIG. 4A shows an overlaid image of cyclosporine A [CsA+Na]⁺ and voclosporin metabolite [VSP+Na]⁺ along with separate images for each parent drug. FIG. 4B shows overlaid and separate images for CsA metabolite [AM4n+Na]⁺ and VSP metabolite [IM4n+Na]⁺. FIG. 4C shows overlaid and separate images for CsA metabolite [AM1/AM1c/AM9+Na]⁺ and VSP metabolite [IM1c/IM4/IM9+Na]±. FIG. 4D shows overlaid and separate images for CsA metabolite [AM19/AM1c9+Na]⁺ and VSP metabolite [IM19 (Cyclic)+Na]⁺. FIG. 4E shows overlaid and separate images for CsA metabolite [AM19/AM1c9+Na]⁺ and VSP metabolite [IM1-Diol-1/IM19+Na]⁺. Mass to signal ratio is visualized as color intensity maps (m/z intensity) to provide spatial information for the detected molecules.

FIG. 5 shows a schematic representation of the long-term safety and tolerability continuation study. As depicted, 216 subjects out of the 357 subjects who were included in the initial 1 year study continued on to the continuation study.

FIG. 6 shows the mean estimated glomerular filtration rates (eGFR) for the 116 subjects in the voclosporin group and the 100 subjects in the control group over a period of 36 months with an additional 4-week follow-up time point. The number of subjects in each study arm over the same time points is also depicted.

FIG. 7 shows the mean urine protein creatinine ratio (UPCR) by visit for the 116 subjects in the voclosporin and the 100 subjects in the control group over a period of 36 months with an additional 4-week follow up time point. The number of subjects in each study arm over the same time points is also depicted.

### DETAILED DESCRIPTION

The present invention is directed to methods, protocols, treatment regimens and/or uses, such as therapeutic uses, that involve the long term use of a calcineurin inhibitor (CNI) while avoiding CNI nephrotoxicity, for example, in the treatment of proteinuric kidney disease, such as lupus nephritis, or associated with a transplant. In some embodiments, the CNI is voclosporin. In some embodiments, the invention involves the administration of a CNI to a subject in need thereof for a prolonged period of time, who is also susceptible to or has an increased risk of acute or chronic CNI nephrotoxicity. In one aspect of the embodiment, the individual requires immunosuppression therapy and presents one or more risk factors for the development of calcineurin inhibitor nephrotoxicity.

In some aspects, the provided methods and uses are based on an observation that treatment with voclosporin in a subject that is in need of CNI therapy, such as a subject with lupus nephritis, results in safe and effective treatment, without a substantial increase in susceptibility to or risk of CNI nephrotoxicity. In some aspects, the results provided herein support that a treatment regimen with voclosporin does not have a substantial impact on the concentrations of urinary electrolytes and/or lipids in the subject. In some aspects, the results provided herein support that a treatment regimen with voclosporin results in a substantial reduction in levels of lipids or a reduction in dyslipidemia, which can be an adverse effect of CNI therapy. The provided methods and uses are also based on an observation from imaging studies that voclosporin and its metabolites do not accumulate at high concentrations in the kidney, whereas cyclosporine, a prominently used CNI, and its metabolites, accumulate in high concentrations, especially in the cortex. Thus, the results described herein support the advantages of using voclosporin and the methods, treatment regimen, protocols and uses provided herein, for subjects that are in need of CNI therapy, particularly for subjects who have elevated susceptibility or risk of CNI nephrotoxicity.

In some aspects, also provided are uses of a calcineurin inhibitor (CNI), such as voclosporin, in the manufacture of a medicament for the treatment of a proteinuric kidney disease, such as lupus nephritis, or nephrotoxicity associated with a transplant. In some aspects, also provided are methods of administering voclosporin for use in treatment of a proteinuric kidney disease, such as lupus nephritis, or nephrotoxicity associated with a transplant or for administration to a subject having a proteinuric kidney disease, such as lupus nephritis, or nephrotoxicity associated with a transplant. In some aspects, the uses of voclosporin are in accord with any of the methods described herein.

Also provided are methods of treatment involving selecting a subject that a proteinuric kidney disease, such as lupus nephritis, or nephrotoxicity associated with a transplant, and administering to the subject voclosporin, for example in accordance with any of the methods or uses described herein. In some embodiments, voclosporin is administered in an effective amount to effect treatment of the proteinuric kidney disease, such as lupus nephritis, or nephrotoxicity associated with a transplant. Uses include uses of voclosporin in such methods and treatments, and in the preparation of a medicament in order to carry out such therapeutic methods. In some embodiments, the methods are carried out by administering voclosporin to the subject having or suspected of having the proteinuric kidney disease, such as lupus nephritis, or nephrotoxicity associated with a transplant, for example, in accordance with the provided embodiments. In some embodiments, the methods thereby treat the proteinuric kidney disease, such as lupus nephritis, or nephrotoxicity associated with a transplant in the subject.

### Calcineurin Inhibitors

Cyclosporine has been a front line CNI used in conjunction with transplant therapy for decades. Another CNI, tacrolimus, has also been used successfully in transplant therapy.

The immunosuppressive properties of cyclosporine and tacrolimus are thought to result from inhibition of calcineurin, a phosphatase. The cyclosporine and tacrolimus are thought to inhibit the phosphatase activity of calcineurin. This inhibition in turn suppresses IL-2 production, and thus T cell activation. Unfortunately, longer term use of these compounds has been reported to cause renal dysfunction, such as a reversible decreases in GFR, now recognized as acute CNI nephrotoxicity, as well as irreversible renal function damage as a result of irreversible and progressive tubule-interstitial injury and glomerulosclerosis or chronic CNI nephrotoxicity.

Voclosporin, described in U.S. Pat. No. 9,765,119, hereby incorporated by reference in its entirety, represents a superior CNI, given its reduced toxicity and ability to be variably dosed to maximize subject tolerance and efficacy. In some embodiments, a subject in need of CNI therapy is administered voclosporin. In some aspects, voclosporin is administered to a subject with active lupus nephritis in combination with background immunosuppressive therapy. In some aspects, voclosporin exhibits a linear pharmacokinetic profile resulting in a consistent dose-concentration relationship. In some aspects, voclosporin provides an advantage of eliminating the need for therapeutic drug monitoring typically associated with use of CNI.

### CNI Therapy and Susceptibility to CNI Nephrotoxicity

CNIs are used to treat subjects suffering from a variety of conditions, particularly those conditions that include an autoimmunity component to the pathology. The immunosuppressant activity of CNIs is thought to help treat the direct causes of symptoms of conditions associated immune system activity. For example, CNIs are used as part of transplant therapy, either alone or with other medications, to prevent transplant rejection particularly in subjects receiving kidney, liver, and heart transplants. CNIs are also used alone or with methotrexate to treat rheumatoid arthritis. CNIs are also used to treat psoriasis. CNIs are also used to treat lupus nephritis. Unfortunately, the long term administration of traditional CNIs in patients with glomerulonephropathy (such as but not limited to lupus nephritis) can lead to additional acute or chronic CNI nephrotoxicity. In general, a subject presenting with or susceptible to developing acute or chronic CNI nephrotoxicity associated with glomerulonephropathy (such as but not limited to autoimmune glomerulonephropathy) will benefit from the present invention.

Subjects presenting with the following may be susceptible to acute CNI nephrotoxicity: acute arteriolopathy tubular vacuolization, and thrombotic microangiopathy (TMA).

Subjects that have received or is a candidate for receiving a transplant, such as an organ transplant or a tissue transplant, and a CNI therapy, for example to reduce or prevent transplant rejection, may be susceptible to acute CNI nephrotoxicity. In some aspects, the subject may have received or is a candidate for receiving a kidney (renal) transplant, a liver transplant, or a heart transplants. In some aspects, the subject may have received or is a candidate for receiving a kidney (renal) transplant.

Representative diagnosis indicating acute CNI nephrotoxicity susceptibility include: renal dysfunction, such as altered renal hemodynamics caused, for example, by other therapeutic compounds; osmotic nephrosis caused by common pharmaceutical excipients such as mannitol, glucose, sucrose, dextran, or by radiocontrast agents; recurrent disease (primary HUS/TTP), ischemia-reperfusion injury, renal infection, and side effects of various other drugs, such as mTOR inhibitors and antiviral agents.

Subject presenting with the following may be susceptible to chronic CNI nephrotoxicity: interstitial fibrosis and tubular atrophy, medial arteriolar hyalinosis, glomerular capsular fibrosis, global glomerulosclerosis, focal segment glomerulosclerosis (FSGS), juxtaglomerular apparatus hyperplasia, and tubular microcalcifications.

Other adverse effects associated with CNI therapy include dyslipidemia, which can contribute to an increased risk of cardiovascular disease. Dyslipidemia can be related to inflammation and the use of immunosuppressants, such as CNI.

Representative diagnosis indicating chronic CNI nephrotoxicity susceptibility include: aging, ischemia-reperfusion injury, infection (UTI, CMV), chronic ischemia, diabetes mellitus, hypertension, glomerular ischemia, bone and mineral imbalance, and proteinuria.

In some aspects, treatment with voclosporin can reduce, prevent, ameliorate or improve CNI nephrotoxicity, such as chronic CNI nephrotoxicity or acute CNI nephrotoxicity. In some aspects, any of the methods, protocols and uses provided herein, can be used for therapeutic purposes to subjects in need of CNI therapy and may be susceptible to or may exhibit increased risk or susceptibility to CNI nephrotoxicity. In some aspects, any of the subjects described herein, for example those who are in need of CNI therapy and may be susceptible to or may exhibit increased risk or susceptibility to CNI nephrotoxicity, may be administered voclosporin according to any of the methods, protocols and uses provided herein.

### Risk Factors for Calcineurin Inhibitor Nephrotoxicity

There are a variety of clinical risk factors for the development of calcineurin inhibitor nephrotoxicity. These include systemic overexposure to cyclosporine and tacrolimus, administration of mTOR inhibitors, the presence of a specific ABCB1 genotype of the kidney, expression of the ABCB1 protein in renal tubular epithelial cells, presence of the CYP3A4/5 genotype, expression of the CYP3A4/5 protein in renal tubular epithelial cells, interaction with other drugs that alter CNI metabolites (e.g., ketoconazole), older kidney age, use of nonsteroidal anti-inflammatory drugs, salt depletion and diuretic use, and genetic polymorphisms of other genes including but not limited to transforming growth factor Beta and angiotensin converting enzyme.

In some embodiments according to any one of the methods described herein, said subject has increased susceptibility to chronic calcineurin inhibitor (CNI) nephrotoxicity. In some embodiments, said subject has increased susceptibility to CNI nephrotoxicity compared to a corresponding population of individuals. In some embodiments, the population of individuals refers to a diseased general population. In some embodiments, the population of individuals refers to general population of a specific gender. In some embodiments, the population of individuals refers to general population of a specific race. In some embodiments, the population of individuals refers to general population of a specific age group. In some embodiments, the population of individuals refers to general population of a specific geographical region. In some embodiments, said subject has increased susceptibility to CNI nephrotoxicity compared to the general population. In some embodiments, said subject has increased susceptibility to CNI nephrotoxicity compared to a diseased general population. In some embodiments, said subject has increased susceptibility to CNI nephrotoxicity compared to a population of the same gender. In some embodiments, said subject has increased susceptibility to CNI nephrotoxicity compared to a population of the same gender. In some embodiments, said subject has increased susceptibility to CNI nephrotoxicity compared to a population of the same race. In some embodiments, said subject has increased susceptibility to CNI nephrotoxicity compared to a population of the same age group. In some aspects, the provided embodiments include assessing or monitoring the risk of CNI nephrotoxicity, for example, by assessing or measuring one or more of the indicators described herein, or changes of the one or more of the indicators described herein.

In some embodiments, wherein said subject has increased susceptibility to chronic calcineurin inhibitor (CNI) nephrotoxicity, said subject exhibits severe renal impairment (CL_{Cr}≥90 mL/min). In some embodiments, said subject exhibits severe renal impairment at a baseline eGFR, wherein the baseline eGFR is any one of about >30, >35, >40, >45, >50, >60 or >70 ml/min/1.73 m². In some embodiments, the voclosporin Cₘₐₓ and/or AUC are increased in subjects with severe renal impairment compared to corresponding subjects without such impairments. In some embodiments, the voclosporin Cₘₐₓ is increased by about any one of 1.1-fold, 1.2-fold, 1.3-fold, 1.4-fold, 1.5-fold, 1.6-fold, 1.7-fold, 1.8-fold, 1.9-fold, 2.0-fold, 3.0-fold, 10.0-fold or more in subjects with severe renal impairment compared to corresponding subjects without such impairments. In some embodiments, the voclosporin AUC is increased by about any one of 1.1-fold, 1.2-fold, 1.3-fold, 1.4-fold, 1.5-fold, 1.6-fold, 1.7-fold, 1.8-fold, 1.9-fold, 2.0-fold, 3.0-fold, 5.0-fold, 10.0-fold or more in subjects with severe renal impairment compared to corresponding subjects without such impairments.

In some embodiments, wherein said subject has increased susceptibility to chronic calcineurin inhibitor (CNI) nephrotoxicity, said subject exhibits hepatic impairment. In some embodiments, said subject exhibits mild hepatic impairment (Child-PughA) or moderate hepatic impairment (Child-PughB). In some embodiments, the voclosporin Cₘₐₓ and/or AUC are increased in subjects with hepatic impairment compared to corresponding subjects without such impairments. In some embodiments, the voclosporin Cₘₐₓ is increased by about any one of 1.1-fold, 1.2-fold, 1.3-fold, 1.4-fold, 1.5-fold, 1.6-fold, 1.7-fold, 1.8-fold, 1.9-fold, 2.0-fold, 3.0-fold, 10.0-fold or more in subjects with mild or moderate hepatic impairment compared to corresponding subjects without such impairments. In some embodiments, the voclosporin AUC is increased by about any one of 1.1-fold, 1.2-fold, 1.4-fold, 1.6-fold, 1.8-fold, 2.0-fold, 2.2-fold, 2.4-fold, 2.6-fold, 2.8-fold, 3.0-fold, 4.0-fold, 5.0-fold, 10.0-fold or more in subjects with mild moderate renal impairment compared to corresponding subjects without such impairments.

Voclosporin is a sensitive CYP3A4 substrate. Co-administration with strong or moderate CYP3A4 inhibitors increases voclosporin exposure. In some embodiments, the voclosporin Cₘₐₓ and/or AUC are increased in subjects receiving strong CYP3A4 inhibitors (e.g., ketoconazole, itraconazole, clarithromycin) or moderate CYP3A4 inhibitors (e.g., verapamil, fluconazole, diltiazem) compared to corresponding subjects without receiving such CYP3A4 inhibitors. In some embodiments, the voclosporin Cₘₐₓ is increased by about any one of 1.1-fold, 1.2-fold, 1.4-fold, 1.6-fold, 1.8-fold, 2.0-fold, 2.2-fold, 2.4-fold, 2.6-fold, 2.8-fold, 3.0-fold, 4.0-fold, 5.0-fold, 10.0-fold or more in subjects receiving strong or moderate CYP3A4 inhibitors compared to corresponding subjects without receiving such CYP3A4 inhibitors. In some embodiments, the voclosporin AUC is increased by about any one of 2.0-fold, 2.2-fold, 2.4-fold, 2.6-fold, 2.8-fold, 3.0-fold, 3.0-fold, 3.2-fold, 3.4-fold, 3.6-fold, 3.8-fold, 4.0-fold, 5.0-fold, 10.0-fold or more in subjects receiving strong or moderate CYP3A4 inhibitors compared to corresponding subjects without receiving such CYP3A4 inhibitors.

In some embodiments according to any one of the methods described herein, the subject suffers from one or more renal diseases. In some embodiments, the subject suffers from one or more renal diseases, wherein the one or more renal diseases contribute to increased susceptibility to chronic calcineurin inhibitor (CNI) nephrotoxicity. In some embodiments, wherein said subject has increased susceptibility to CNI nephrotoxicity, said subject exhibits one or more forms of glomerulonephropathy. In some embodiments, wherein said subject has increased susceptibility to CNI nephrotoxicity, and wherein said subject exhibits one or more forms of glomerulonephropathy, prolonged administration of traditional CNI (such as cyclosporine or tacrolimus) can result in long term renal damage, such as but not limited to additional acute and chronic nephrotoxicity. In some embodiments, the subject exhibits glomerulonephropathy. In some embodiments, the subject exhibits chronic glomerulonephropathy. In some embodiments, the subject exhibits inflammatory glomerulonephropathy. In some embodiments, the subject exhibits non-inflammatory glomerulonephropathy. In some embodiments, the subject exhibits autoimmune glomerulonephropathy. In some embodiments, the subject exhibits acute glomerulonephropathy. In some embodiments, the subject exhibits glomerulonephritis. In some embodiments, the subject exhibits chronic glomerulonephritis. In some embodiments, the subject exhibits acute glomerulonephritis. In some embodiments, the subject exhibits an inflammatory renal disease. In some embodiments, the subject exhibits a non-inflammatory renal disease. In some embodiments, the subject exhibits an inflammatory renal disease in the context of autoimmunity. In some embodiments, the autoimmune renal disease may result from a self-antigen within renal tissue (such as Goodpasture antigen). In some embodiments, the autoimmune renal disease may result from a self-antigens residing outside the kidney that causes immune-complexes within the kidneys, triggering tissue damage events (e.g. lupus nephritis). In some embodiments, the autoimmune renal disease may result from antigen and antibodies that are neither derived nor deposited within the kidneys, but wherein interaction of antibodies with antigens or with antigen-bearing cells cause the disease (e.g. autoantibodies to neutrophil cytoplasmic antigens (ANCA)-associated vasculitis or glomerulonephritis). In some embodiments, the subject suffers from anti-glomerular base membrane disease (anti-GBM disease). In some embodiments, the subject suffers from lupus nephritis. In some embodiments, the subject suffers from systemic lupus erythematosus (SLE). In some aspects, up to half of subjects with systemic lupus erythematosus (SLE) can develop LN, which can result in severe and permanent damage to the kidneys and, in some cases, renal failure. In some embodiments, the subject suffers from ANCA-associated vasculitis. In some embodiments, the subject suffers from ANCA-associated glomerulonephritis.

In some embodiments, wherein said subject has increased susceptibility to chronic calcineurin inhibitor (CNI) nephrotoxicity, said subject exhibits one or more of: (a) variability in P-glycoprotein expression and/or activity; (b) variability in CYP3A4/5 expression and/or activity, (c) older kidney age; (d) salt depletion; (e) the use of nonsteroidal anti-inflammatory drugs; and/or (f) genetic polymorphisms in TGF-β and/or ACE gene. In some embodiments, said subject exhibits upregulation of local renal P-glycoprotein. In some embodiments, said subject exhibits about any one of 20%, 40%, 50%, 75%, 100%, 2-fold, 5-fold, 10-fold, 20-fold, 50-fold, 75-fold, 100-fold, 500-fold, 1000-fold or more upregulation of local renal P-glycoprotein compared to a corresponding population described above. In some embodiments, said subject exhibits one or more single nucleotide polymorphisms in ABCB1 gene. In some embodiments, said subject exhibits the TT genotype at position 3435 of the ABCB1 gene. In some embodiments, said subject exhibits the TT genotype at position 3435 of the ABCB1 gene in a grafted kidney. In some embodiments, said subject does not exhibits the TT genotype at position 2677 of the ABCB1 gene. In some embodiments, said subject does not exhibit the TT genotype at position 2677 of the ABCB1 gene in a grafted kidney. In some embodiments, said subject has been exposed to or has been administered nonsteroidal anti-inflammatory drugs. In some embodiments, said subject exhibits an older kidney age, such as about any one of 1, 2, 3, 4, 5, 8, 10, 12, 15, 18, 20, 25, 30, 35, 40 older kidney age than a corresponding population described above. In some embodiments, said subject exhibits salt depletion, such as up to 10%, 20%, 30%, 40%, 50%, 60%, 70%, 80% or more depletion as compared to a corresponding population described above. In some embodiments, said subject exhibits salt depletion as a result of dietary sodium restriction. In some embodiments, salt depletion in said subject results in increased renal tubular reabsorption of drugs, such as about any one of 20%, 40%, 50%, 75%, 100%, 2-fold, 5-fold, 10-fold, 20-fold, 50-fold, 75-fold, 100-fold, 500-fold, 1000-fold or more drug reabsorption of drugs as compared to a corresponding population described above. In some embodiments, said subject exhibits one or more polymorphisms in CYP3A4 and/or CYP3A5 gene. In some embodiments, said subject is a CYP3A5*1 carrier. In some embodiments, the hepatic and intestinal CYP3A5 of said subject are expressed from CYP3A5*1. In some embodiments, said subject is a CYP3A5*1 carrier, and generation of tacrolimus metabolite in the subject liver is any one of 20%, 40%, 50%, 75%, 100%, 2-fold, 5-fold, 10-fold, 20-fold, 50-fold, 75-fold, 100-fold, 500-fold, 1000-fold or more higher than that of a corresponding population described above. In some embodiments, said subject exhibits genetic polymorphisms in TGF-β and/or Angiotensin converting enzyme (ACE). In some embodiments, said subject has increased ACE activity, such as about any one of 20%, 40%, 50%, 75%, 100%, 2-fold, 5-fold, 10-fold, 20-fold, 50-fold, 75-fold, 100-fold, 500-fold, 1000-fold or more increased activity. In some embodiments, said subject has increased serum levels of ACE, such as about any one of 20%, 40%, 50%, 75%, 100%, 2-fold, 5-fold, 10-fold, 50-fold, 75-fold, 100-fold, 500-fold, 1000-fold or more increased serum ACE levels. In some embodiments, said subject exhibits TGF-β polymorphism in codon 10. In some embodiments, said subject exhibits increased local TGF-β production in allograft. In some embodiments, said subject has upregulation of TGF-β in tubular epithelial cells, such as about any one of TGF-β 20%, 40%, 50%, 75%, 100%, 2-fold, 5-fold, 10-fold, 20-fold, 50-fold, 75-fold, 100-fold, 500-fold, 1000-fold or more upregulation of TGF-β in tubular epithelial cells.

### Method to Reduce CNI Nephrotoxicity in Treatment of a Proteinuric Kidney Disease or a Transplant

In some aspects, provided are method to reduce chronic calcineurin inhibitor nephrotoxicity in treatment of a proteinuric kidney disease or a transplant, such as an organ transplant, for example a kidney (renal) transplant, a heart transplant, a liver transplant, or a heart transplant.

In some embodiments, the method to reduce chronic calcineurin inhibitor nephrotoxicity in treatment of a proteinuric kidney disease or associated with a transplant disclosed herein comprises administering to a subject diagnosed with said disease a predetermined daily dosage of effective amounts of voclosporin over a projected treatment period of at least 55 weeks. In some embodiments, the method further includes assessing the estimated Glomerular Filtration Rate (eGFR) of the subject at at least a first time point and a second time point on different days of said treatment period, and if the eGFR of said subject decreases by more than a target % to below a predetermined value, between said first and second time points, reducing the daily dosage by increment(s) of 7.9 mg BID or stopping the administering of voclosporin to said subject. In some examples, the method further includes assessing the estimated Glomerular Filtration Rate (eGFR) of the subject at at least a first time point and a second time point on different days of said treatment period, and if the eGFR of the subject decreases by less than said target %, between said first and second time points, the method includes continuing administering the same predetermined daily dosage of voclosporin to said subject. In some embodiments, the treatment period is at least any one of about 50, 55, 65, 70, 75, 80, 85, 90, 95, 100, 110, 120 weeks, or any length of treatment periods there between.

In some embodiments, the method to reduce chronic calcineurin inhibitor nephrotoxicity in treatment of a proteinuric kidney disease or associated with a transplant disclosed herein comprises administering to a subject diagnosed with lupus nephritis a predetermined daily dosage of effective amounts of voclosporin over a projected treatment period of at least 55 weeks. In some embodiments, the method further includes assessing the estimated Glomerular Filtration Rate (eGFR) of the subject at at least a first time point and a second time point on different days of said treatment period, and if the eGFR of said subject decreases by more than a target % to below a predetermined value, between said first and second time points, reducing the daily dosage by increment(s) of 7.9 mg BID or stopping the administering of voclosporin to said subject. In some examples, the method further includes assessing the estimated Glomerular Filtration Rate (eGFR) of the subject at at least a first time point and a second time point on different days of said treatment period, and if the eGFR of the subject decreases by less than said target %, between said first and second time points, the method includes continuing administering the same predetermined daily dosage of voclosporin to said subject. In some embodiments, the treatment period is at least any one of about 50, 55, 65, 70, 75, 80, 85, 90, 95, 100, 110, 120 weeks, or any length of treatment periods there between.

In some embodiments, the first time point is immediately preceding initiating said protocol. In some examples, the predetermined value is in the range of about 30 to about 110 ml/min/1.73 m². In some embodiments, the predetermined value is in the range of about 50 to about 90 ml/min/1.73 m². In some embodiments, the predetermined value is in the range of about any one of 30 to 40, 40 to 50, 50 to 60, 60 to 70, 70 to 80, 80 to 90, 90 to 100 and 100-110 ml/min/1.73 m². In some embodiments, the predetermined value is any one of about 30, 40, 50, 70, 80, 90, or 100 ml/min/1.73 m². In some embodiments, the predetermined value is about ml/min/1.73 m². In some embodiments, the target % is in the range of about 10% to about 60%. In some embodiments, the target % is in the range of about 20% to about 45%. In some embodiments, the target % is in the range of about any one of 10% to 20%, 20% to 30%, 30% to 35%, 35% to 40%, 40% to 45%, 45% to 50%, 50% to 55% and 55% to 60%. In some embodiments, the target % is approximately 20%. In some embodiments, the target % is approximately 30%.

In some embodiments, the method to reduce chronic calcineurin inhibitor nephrotoxicity in treatment of a proteinuric kidney disease or associated with a transplant disclosed herein comprises administering to a subject diagnosed with lupus nephritis a predetermined daily dosage of effective amounts of voclosporin over a projected treatment period of at least 55 weeks. In some embodiments, the method further includes assessing the estimated Glomerular Filtration Rate (eGFR) of the subject at at least a first time point and a second time point on different days of said treatment period, and if the eGFR of said subject decreases by more than a 20% to below 60 ml/min/1.73 m², between said first and second time points, reducing the daily dosage by increment(s) of 7.9 mg BID or stopping the administering of voclosporin to said subject. In some examples, the method further includes assessing the estimated Glomerular Filtration Rate (eGFR) of the subject at at least a first time point and a second time point on different days of said treatment period, and if the eGFR of the subject decreases by less than 20%, between said first and second time points, the method includes continuing administering the same predetermined daily dosage of voclosporin to said subject.

In some embodiments, wherein the method comprises assessing the estimated Glomerular Filtration Rate (eGFR) of the subject at at least a first time point and a second time point on different days of said treatment period, the first time point is immediately preceding initiating said protocol. In some embodiment, the method further comprises assessing eGFR of the subject every two weeks subsequent to initiating said protocol. In some embodiments, the method comprises assessing eGFR of the subject every two weeks in the first month, and every four weeks thereafter, subsequent to initiating said protocol. In some embodiments, wherein eGFR of said subject decreases by more than a target % to below a predetermined value at any assessment subsequent to initiating said protocol, the method comprises reducing the daily dosage by increment(s) of 7.9 mg BID or stopping the administering of voclosporin to said subject.

In some embodiments, wherein the method comprises assessing the eGFR of the subject at at least a first time point and a second time point on different days of said treatment period, the first time point is immediately preceding initiating said protocol. In some embodiments, wherein eGFR of said subject decreases by more than a target % compared to eGFR at the first time point to below a predetermined value at an assessment subsequent to initiating said protocol, the method comprises reducing the daily dosage by 7.9 mg BID. In some embodiments, wherein eGFR of said subject decreases by more than the target % compared to eGFR at the first time point to below the predetermined value at an assessment subsequent to initiating said protocol, and wherein the daily dosage is reduced by 7.9 mg BID, the method comprises assessing eGFR of the subject two weeks subsequent to dosage reduction, and further comprises a second reduction of daily dosage by 7.9 mg BID if eGFR of said subject decreases by more than the target % compared to eGFR at the first time point to the predetermined value in the assessment two weeks subsequent to the first dosage reduction. In some embodiments, wherein the method comprises one or more dosage reductions, the method comprises assessing eGFR of the subject every two weeks subsequent to the one or more dosage reductions, and comprises increasing the daily dosage by 7.9 mg BID for each eGFR assessment where eGFR of said subject decreases by less than the target % compared to eGFR at the first time point. In some embodiments, the dosage increase does not cause the dosage to exceed the initial predetermined dosage. In some examples, the predetermined value is in the range of about to about 110 ml/min/1.73 m². In some embodiments, the predetermined value is in the range of about 50 to about 90 ml/min/1.73 m². In some embodiments, the predetermined value is in the range of about any one of 30 to 40, 40 to 50, 50 to 60, 60 to 70, 70 to 80, 80 to 90, 90 to 100 and 100-110 ml/min/1.73 m². In some embodiments, the predetermined value is any one of about 30, 50, 60, 70, 80, 90, or 100 ml/min/1.73 m². In some embodiments, the predetermined value is about 60 ml/min/1.73 m². In some embodiments, the target % is in the range of about 10% to about 60%. In some embodiments, the target % is in the range of about 10% to about 45%. In some embodiments, the target % is in the range of about any one of 10% to 20%, 20% to 30%, 30% to 35%, 35% to 40%, 40% to 45%, 45% to 50%, 50% to 55% and 55% to 60%. In some embodiments, the target % is approximately 20%.

In some embodiments, wherein eGFR of said subject decreases by between about 20% to about 30% compared to eGFR at the first time point to below 60 ml/min/1.73 m² at an assessment subsequent to initiating said protocol, the method comprises reducing the daily dosage by 7.9 mg BID. In some embodiments, wherein eGFR of said subject decreases by between about 20% to about 30% compared to eGFR at the first time point, to below 60 ml/min/1.73 m² at an assessment subsequent to initiating said protocol, and wherein the daily dosage is reduced by 7.9 mg BID, the method comprises assessing eGFR of the subject two weeks subsequent to dosage reduction, and further comprises a second reduction of daily dosage by 7.9 mg BID if eGFR of said subject decreases by more than 20% compared to eGFR at the first time point to below 60 ml/min/1.73 m² in the assessment two weeks subsequent to the first dosage reduction. In some embodiments, wherein the method comprises one or more dosage reductions, the method comprises assessing eGFR of the subject every two weeks subsequent to the one or more dosage reductions, and comprises increasing the daily dosage by 7.9 mg BID for each eGFR assessment where eGFR of said subject decreases by less than 20% compared to eGFR at the first time point. In some embodiments, the dosage increase does not cause the dosage to exceed the initial predetermined dosage.

In some embodiments, wherein the method comprises assessing the eGFR of the subject at at least a first time point and a second time point on different days of said treatment period, the first time point is immediately preceding initiating said protocol. In some embodiments, wherein eGFR of said subject decreases by equal to or more than a first target % compared to eGFR at the first time point to below a predetermined value at any assessment subsequent to initiating said protocol, the method comprises stopping the administering of voclosporin to said subject. In some embodiments, wherein eGFR of said subject decreases by equal or more than the target % compared to eGFR at the first time point to below the predetermined value at an assessment subsequent to initiating said protocol, and wherein the administration of voclosporin to said subject is stopped, the method comprises assessing eGFR of the subject two weeks subsequent to stopping administration, and further comprises re-initiating the administration at the initial predetermined dosage if eGFR of said subject decreases by less than a second target % compared to eGFR at the first time point in the assessment two weeks subsequent to the stopping of administration of voclosporin. In some embodiments, the predetermined value is in the range of about 50 to about 90 ml/min/1.73 m². In some embodiments, the predetermined value is in the range of about any one of 30 to 40, 40 to 50, 50 to 60, 60 to 70, 70 to 80, 80 to 90, 90 to 100 and 100-110 ml/min/1.73 m². In some embodiments, the predetermined value is any one of about 30, 40, 50, 60, 70, 80, 90, or 100 ml/min/1.73 m². In some embodiments, the predetermined value is about 60 ml/min/1.73 m². In some embodiments, the first target % is in the range of about 10% to about 60%. In some embodiments, the first target % is in the range of about 20% to about 45%. In some embodiments, the first target % is in the range of about any one of 10% to 20%, 20% to 30%, 30% to 35%, 35% to 40%, 40% to 45%, 45% to 50%, 50% to 55% and 55% to 60%. In some embodiments, the first target % is approximately 30%. In some embodiments, the second target % is in the range of about 10% to about 60%. In some embodiments, the second target % is in the range of about 10% to about 45%. In some embodiments, the second target % is in the range of about any one of 10% to 20%, 20% to 30%, 30% to 35%, 35% to 40%, 40% to 45%, 45% to 50%, 50% to 55% and 55% to 60%. In some embodiments, the second target % is approximately 20%.

In some embodiments, wherein eGFR of said subject decreases by equal to or more than 30% compared to eGFR at the first time point to below 60 ml/min/1.73 m² at any assessment subsequent to initiating said protocol, the method comprises stopping the administering of voclosporin to said subject. In some embodiments, wherein eGFR of said subject decreases by equal or more than 30% compared to eGFR at the first time point to below 60 ml/min/1.73 m² at an assessment subsequent to initiating said protocol, and wherein the administration of voclosporin to said subject is stopped, the method comprises assessing eGFR of the subject two weeks subsequent to stopping administration, and further comprises re-initiating the administration at the initial predetermined dosage if eGFR of said subject decreases by less than 20% compared to eGFR at the first time point in the assessment two weeks subsequent to the stopping of administration of voclosporin.

In some embodiments, the method disclosed herein further includes identifying a subject as appropriate for said method prior to conducting said method on the subject by (a) determining that the urine protein creatinine ratio (UPCR) of the subject is >1 mg/mg as measured by first morning void or 24 hour urine, and (b) determining that the subject has an eGFR as measured by Chronic Kidney Disease Epidemiology Collaboration equation (CKD-EP1) of >45 ml/min/1.73 m²; if the conditions of (a) and (b) are met, the subject is identified as appropriate for said method. In some embodiments, the method comprises determining that the urine protein creatinine ratio (UPCR) of the subject is >1.5 mg/mg as measured by first morning void or 24 hour urine.

In some embodiments, the method disclosed herein further includes assessing the interstitial fibrosis and tubular atrophy of said subject by renal biopsies at at least a first time point and a second time point on different days of said treatment period, and if interstitial fibrosis and tubular atrophy is observed in a more than a predetermined percentage of cortical area, between said first and second time points, reducing the daily dosage by increment(s) of 7.9 mg BID or stopping the administering of voclosporin to said subject. In some embodiments, the method disclosed herein further includes assessing the interstitial fibrosis and tubular atrophy of said subject by renal biopsies at at least a first time point and a second time point on different days of said treatment period, and if interstitial fibrosis and tubular atrophy is observed in less than a predetermined percentage in cortical area, between said first and second time points, continuing administering the same predetermined daily dosage of voclosporin to said subject. In some embodiments, the predetermined percentage is any one of 0%, 1%, 2%, 5%, 10%, 15%, 20%, 25%, or 50%, or any percentage there between. In some embodiments, the predetermined percentage is any one of about 0% to 5%, about 5% to about 25%, about 25% to about 50%, or about 50% to about 99%. In some embodiments, the predetermined percentage is any one of ≤5%; 5-25%; 25-≤50%; or >50%. In some embodiments, the predetermined percentage is about 5%. In some embodiments, interstitial fibrosis and tubular atrophy is determined by involved by mononuclear cell infiltrate in non-scarred cortical area, and are analogous to the Banff interstitial inflammation scores for kidney allograft rejection. In some embodiments, the percentage of non-scarred cortical area involved by mononuclear cell infiltrate is assessed by hematoxylin and eosin staining (H&E), periodic acid-Schiff (PAS), silver and trichrome stains and collagen III immunohistochemistry and are graded semi-quantitatively using the following categories: Grade 0: ≤5%; Grade 1: 5-25%; Grade 2: 25-≤50%; Grade 3: >50%.

In some embodiments, the method disclosed herein further includes assessing the presence of medial arteriolar hyalinosis of said subject by renal biopsies at at least a first time point and a second time point on different days of said treatment period, and if medial arteriolar hyalinosis is present, between said first and second time points, reducing the daily dosage by increment(s) of 7.9 mg BID or stopping the administering of voclosporin to said subject. In some embodiments, the method disclosed herein further includes assessing the presence of medial arteriolar hyalinosis of said subject by renal biopsies at at least a first time point and a second time point on different days of said treatment period, and if medial arteriolar hyalinosis is not present, between said first and second time points, continuing administering the same predetermined daily dosage of voclosporin to said subject. In some embodiments, medial arteriolar hyalinosis is identified by the replacement of necrotic smooth muscle cells with focal, circular lumpy protein (hyaline) deposits at the periphery of the wall of afferent arterioles, and the narrowing of the vascular lumen. In some embodiments, the medial arteriolar hyalinosis is identified by the replacement of necrotic smooth muscle cells with focal, circular lumpy protein (hyaline) deposits at the periphery of the wall of afferent arterioles, or the narrowing of the vascular lumen.

In some embodiments, the method disclosed herein further includes assessing the presence of glomerular injury of said subject by renal biopsies at at least a first time point and a second time point on different days of said treatment period, and if glomerular injury is present, between said first and second time points, reducing the daily dosage by increment(s) of 7.9 mg BID or stopping the administering of voclosporin to said subject. In some embodiments, the method disclosed herein further includes assessing the presence of glomerular injury of said subject by renal biopsies at at least a first time point and a second time point on different days of said treatment period, and if glomerular injury is not present, between said first and second time points, continuing administering the same predetermined daily dosage of voclosporin to said subject. In some aspects, the glomerular injury comprises global and segmental glomerulosclerosis, tubular atrophy, interstitial fibrosis, and arteriosclerosis. In some embodiments, the glomerular injury comprises global and segmental glomerulosclerosis, tubular atrophy, interstitial fibrosis, or arteriosclerosis. In some examples, the glomerular injury is present when total renal chronicity score is >1. To determine glomerular injury, global and segmental glomerulosclerosis is scored from 0 to 3, tubular atrophy from 0 to 3, interstitial fibrosis from 0 to 3 and arteriosclerosis from 0 to 1. The scores are then added (total renal chronicity score) to grade the overall severity of the chronic lesions into minimal (0-1 total score), mild (2-4 total score), moderate (5-7 total score) and severe (≥8 total score). In some embodiments, the glomerular injury is determined to be present when total renal chronicity score is greater than any one of: 0, 1, 2, 3, 4, 5, 6, 7, 8. In some embodiments, the glomerular injury is determined to be present when total renal chronicity score is any one of: about 1, about 2 to about 4, about 5 to about 7, or about 8 to about 10. In some embodiments, the glomerular injury is a mild glomerular injury. In some embodiments, the glomerular injury is a moderate glomerular injury. In some embodiments, the glomerular injury is a severe glomerular injury.

In some embodiments, the method disclosed herein further includes assessing the presence of juxtaglomerular apparatus (JGA) hyperplasia of said subject by renal biopsies at at least a first time point and a second time point on different days of said treatment period, and if JGA hyperplasia is present, between said first and second time points, reducing the daily dosage by increment(s) of 7.9 mg BID or stopping the administering of voclosporin to said subject. In some embodiments, the method disclosed herein further includes assessing the presence of juxtaglomerular apparatus (JGA) hyperplasia of said subject by renal biopsies at at least a first time point and a second time point on different days of said treatment period, and if JGA hyperplasia is not present, between said first and second time points, continuing administering the same predetermined daily dosage of voclosporin to said subject. In some examples, JGA hyperplasia includes enlargements of juxtaglomerular apparatus components comprising one or more of: the vascular components, the mesangial cell components, the tubular components (the macula densa), and the presence of intracellular renin granules. In some examples, the JGA hyperplasia includes enlargements of juxtaglomerular apparatus components comprising one or more of: the vascular components, the mesangial cell components, the tubular components (the macula densa), or the presence of intracellular renin granules.

In some embodiments, the method disclosed herein further includes assessing the presence of tubular microcalcifications of said subject by renal biopsies at at least a first time point and a second time point on different days of said treatment period, and if tubular microcalcifications is present, between said first and second time points, reducing the daily dosage by increment(s) of 7.9 mg BID or stopping the administering of voclosporin to said subject. In some embodiments, the method disclosed herein further includes assessing the presence of tubular microcalcifications of said subject by renal biopsies at at least a first time point and a second time point on different days of said treatment period, and if tubular microcalcifications is not present, between said first and second time points, continuing administering the same predetermined daily dosage of voclosporin to said subject.

In some embodiments, the method disclosed herein further includes assessing the P-glycoprotein (P-gp) expression of said subject by renal biopsies at at least a first time point and a second time point on different days of said treatment period, and if loss of expression of P-gp is more than a predetermined value, between said first and second time points, reducing the daily dosage by increment(s) of 7.9 mg BID or stopping the administering of voclosporin to said subject. In some embodiments, the method disclosed herein further includes assessing the P-glycoprotein (P-gp) expression of said subject by renal biopsies at at least a first time point and a second time point on different days of said treatment period, and if loss of expression of P-gp is less than the predetermined value, between said first and second time points, continuing administering the same predetermined daily dosage of voclosporin to said subject. In some examples, the predetermined value for loss of expression of P-gp is 10% loss of P-gp expression in tubules in the cortical area. P-glycoprotein expression in glomeruli and tubular epithelial cells can be scored semi-quantitatively according to the intensity and distribution. The classification can include: normal=0 point, P-gp expression loss in <10% of tubules placed in the cortical area; mild loss=1 point, P-gp expression loss in 10% to 24% of tubuli in the cortical area; moderate loss=2 points, P-gp expression loss in 25% to 50% of tubuli in the cortical area; severe loss=3 points, P-gp expression loss in >50% of tubuli in the cortical area. In some embodiments, the predetermined value for loss of expression of P-gp is about 5% to about 50% loss of P-gp expression in tubules in the cortical area. In some embodiments, the predetermined value for loss of expression of P-gp is about any one of: 5%, 8%, 10%, 15%, 20%, 25%, 30%, 35%, 40%, 45%, or 50% loss of P-gp expression in tubules in the cortical area. In some embodiments, the predetermined value for loss of expression of P-gp is any one of: about 1% to about 5%, about 5% to about 10%, about 10% to about 25%, about 25% to about 50%, or 50% or more loss of P-gp expression in tubules in the cortical area. In some examples, the predetermined value for loss of expression of P-gp is 10% loss of P-gp expression in tubules in the cortical area.

In some embodiments, the method disclosed herein further includes assessing the Calcineurin Inhibitor (CNI) Nephrotoxicity and/or Banff Scores of said subject by renal biopsies at at least a first time point and a second time point on different days of said treatment period, and if the CNI Nephrotoxicity and/or Banff Scores are outside of predetermined ranges, between said first and second time points, reducing the daily dosage by increment(s) of 7.9 mg BID or stopping the administering of voclosporin to said subject. In some embodiments, the method disclosed herein further includes assessing the Calcineurin Inhibitor (CNI) Nephrotoxicity and/or Banff Scores of said subject by renal biopsies at at least a first time point and a second time point on different days of said treatment period, and if the CNI Nephrotoxicity and/or Banff Scores are within predetermined ranges, between said first and second time points, continuing administering the same predetermined daily dosage of voclosporin to said subject. In some embodiments, the predetermined range of CNI Nephrotoxicity Score is about any one of 0 to 1, 0 to 2, 0 to 3, 0 to 4, 0 to 5, 0 to 6, 0 to 7, 0 to 8, 0 to 9, or 0 to 10. In some embodiments, the predetermined range of CNI Nephrotoxicity Score is about any one of <1, <2, <3, <4, <5, <6, <7, <8, <9, or <10. In some embodiments, the predetermined range of Banff Score is about any one of 0 to 1, 0 to 2, 0 to 3, 0 to 4, 0 to 5, 0 to 6, 0 to 7, 0 to 8, 0 to 9, or 0 to 10. In some embodiments, the predetermined range of Banff Score is about any one of <1, <2, <3, <4, <5, <6, <7, <8, <9, or <10. In some embodiments, the predetermined range of CNI Nephrotoxicity Score is 0-3, or the predetermined range of Banff Score is 0-3.

In some embodiments, the method disclosed herein further includes assessing one or more of the National Institutes of Health Activity Index (NIH-AI), the National Institutes of Health Chronicity Index (NIH-CI), and the Tubulointerstitial Activity Index (TIAI) of said subject by renal biopsies at at least a first time point and a second time point on different days of said treatment period, and if the NIH-AI, NIH-CI, and/or TIAI are outside of predetermined ranges, between said first and second time points, reducing the daily dosage by increment(s) of 7.9 mg BID or stopping the administering of voclosporin to said subject. In some embodiments, the method disclosed herein further includes assessing one or more of the National Institutes of Health Activity Index (NIH-AI), the National Institutes of Health Chronicity Index (NIH-CI), and the Tubulointerstitial Activity Index (TIAI) of said subject by renal biopsies at at least a first time point and a second time point on different days of said treatment period, and if the NIH-AI, NIH-CI, and/or TIAI are within predetermined ranges, between said first and second time points, continuing administering the same predetermined daily dosage of voclosporin to said subject. In some embodiments, the predetermined range of NIH-AI is about any one of 0 to 1, 0 to 2, 0 to 3, to 4, 0 to 5, 0 to 6, 0 to 7, 0 to 8, 0 to 9, 0 to 10, 0 to 11, 0 to 12, 0 to 13, 0 to 14, 0 to 15, 0 to 16, 0 to 17, 0 to 18, 0 to 19 or 0 to 20. In some embodiments, the predetermined range of NIH-AI is any one of <1, <2, <3, <4, <5, <6, <7, <8, <9, <10, <11, <12, <13, <14, <15, <16, <17, <18, <19, or <20. In some embodiments, the predetermined range of NIH-AI is about 0-6. In some embodiments, the predetermined range of NIH-CI is about any one of 0 to 1, 0 to 2, 0 to 3, to 4, 0 to 5, 0 to 6, 0 to 7, 0 to 8, 0 to 9, or 0 to 10. In some embodiments, the predetermined range of NIH-CI is any one of <1, <2, <3, <4, <5, <6, <7, <8, <9, or <10. In some embodiments, the predetermined range of NIH-CI is about 0-3. In some embodiments, the predetermined range of TIAI is about any one of 0 to 1, 0 to 2, 0 to 3, 0 to 4, 0 to 5, 0 to 6, 0 to 7, 0 to 8, 0 to 9, 0 to 0 to 11, 0 to 12, 0 to 13, 0 to 14, or 0 to 15. In some embodiments, the predetermined range of NIH-AI is any one of <1, <2, <3, <4, <5, <6, <7, <8, <9, <10, <11, <12, <13, <14, or <15. In some embodiments, the predetermined range of TIAI is about 0-5. In some embodiments, the predetermined range of NIH-AI is about 0-6, or the predetermined range of NIH-CI is about 0-3, and/or the predetermined range of TIAI is about 0-5. In some embodiments, the predetermined range of NIH-AI is 0-6, and the predetermined range of NIH-CI is 0-3, or the predetermined range of TIAI is 0-5.

In some embodiments, the method disclosed herein further includes assessing urine anion gap (UAG) in urinary specimens of said subject at at least a first time point and a second time point on different days of said treatment period, and if the UAG is outside of a predetermined range, between said first and second time points, reducing the daily dosage by increment(s) of 7.9 mg BID or stopping the administering of voclosporin to said subject. In some embodiments, the method disclosed herein further includes assessing urine anion gap (UAG) in urinary specimens of said subject at at least a first time point and a second time point on different days of said treatment period, and if the UAG is within the predetermined range, between said first and second time points, continuing administering the same predetermined daily dosage of voclosporin to said subject. Urinary specimens can be assessed with NH4+ loading test involving administration of ammonium chloride (NH4C1) in a quantity sufficient to acidify urine. Urinary pH is calculated using the urine anion gap (UAG) as a surrogate marker for NH4+ secretion. The UAG is derived by subtracting urine chloride (Cl−) from sodium (Na+) and potassium (K+) ions present in the urine. In some embodiments, the predetermined range of UAG is between about 5 to about 150 mEq/L. In some embodiments, the predetermined range of UAG is between any one of about 10 to about 120, about 15 to about 100, about 20 to about about 30 to about 80, about 40 to about 70, about 50 to about 60, about 10 to about 90, about to about 60, about 20 to about 100, about 20 to about 90, about 20 to about 60, about 30 to about 120, about 30 to about 100, about 30 to about 90, about 30 to about 60 mEq/L. In some embodiments, the predetermined range of UAG is between about 20 to about 90 mEq/L.

In some embodiments, the method disclosed herein further includes assessing hyperkalemia, hypomagnesemia, magnesium wasting and hyperuricemia in serum and urine samples of said subject at at least a first time point and a second time point on different days of said treatment period, and if hyperkalemia, hypomagnesemia, magnesium wasting and hyperuricemia are detected, between said first and second time points, reducing the daily dosage by increment(s) of 7.9 mg BID or stopping the administering of voclosporin to said subject. In some embodiments, the method disclosed herein further includes assessing hyperkalemia, hypomagnesemia, magnesium wasting and/or hyperuricemia in serum and urine samples of said subject at at least a first time point and a second time point on different days of said treatment period, and if hyperkalemia, hypomagnesemia, magnesium wasting or hyperuricemia are detected, between said first and second time points, reducing the daily dosage by increment(s) of 7.9 mg BID or stopping the administering of voclosporin to said subject. In some embodiments, the method disclosed herein further includes assessing hyperkalemia, hypomagnesemia, magnesium wasting and hyperuricemia in serum and urine samples of said subject at at least a first time point and a second time point on different days of said treatment period, and if hyperkalemia, hypomagnesemia, magnesium wasting and hyperuricemia are not detected, between said first and second time points, continuing administering the same predetermined daily dosage of voclosporin to said subject. In some embodiments, the method disclosed herein further includes assessing hyperkalemia, hypomagnesemia, magnesium wasting and hyperuricemia in serum and urine samples of said subject at at least a first time point and a second time point on different days of said treatment period, and if hyperkalemia, hypomagnesemia, magnesium wasting or hyperuricemia are not detected, between said first and second time points, continuing administering the same predetermined daily dosage of voclosporin to said subject. In some embodiments, hyperkalemia is determined by a serum potassium level of more than about any one of 4, 5, 6, 7, 8, 9, or 10 mmol/L. In some embodiments, hyperkalemia is determined by a serum potassium level of more than about 5 mmol/L. In some aspects, hypomagnesemia is determined by a serum magnesium level less than about any one of 0.9, 1.1, 1.2, 1.3, 1.4, 1.5, 1.6, 1.7, 1.8, 1.9, 2.0, 2.1, 2.2, 2.3, 2.4 or 2.5 mg/dL. In some aspects, hypomagnesemia is determined by a serum magnesium level less than about 1.4 mg/dL. In some aspects, magnesium wasting is determined by a urine magnesium level of more than about any one of 0.8, 1.0, 1.2, 1.4, 1.6, 1.8, 2, 2.2, 2.4, 2.6, 2.8, 3.0, 3.5 or 4.0 mEq. In some aspects, magnesium wasting is determined by a urine magnesium level of more than about 2 mEq. In some aspects, hyperuricemia is determined by a serum uric acid level of more than about any one of 4.0, 4.5, 5.0, 5.5, 6.0, 6.5, 7.0, 7.5, 8.0, 8.5, 9.0, 9.5 or 10.0 mg/dL. In some aspects, hyperuricemia is determined by a serum uric acid level of more than about 7.0 mg/dL.

In some embodiments, the method disclosed herein further includes assessing serum creatinine (SCr) and serum cystatin C (SCysC) in serum samples of said subject at at least a first time point and a second time point on different days of said treatment period, and if the SCr and SCysC levels are elevated above predetermined ranges, between said first and second time points, reducing the daily dosage by increment(s) of 7.9 mg BID or stopping the administering of voclosporin to said subject. In some embodiments, the method disclosed herein further includes assessing serum creatinine (SCr) and serum cystatin C (SCysC) in serum samples of said subject at at least a first time point and a second time point on different days of said treatment period, and if the SCr or SCysC levels are elevated above predetermined ranges, between said first and second time points, reducing the daily dosage by increment(s) of 7.9 mg BID or stopping the administering of voclosporin to said subject. In some embodiments, the method disclosed herein further includes assessing serum creatinine (SCr) and serum cystatin C (SCysC) in serum samples of said subject at at least a first time point and a second time point on different days of said treatment period, and if the SCr and SCysC levels are within predetermined ranges, between said first and second time points, continuing administering the same predetermined daily dosage of voclosporin to said subject. In some embodiments, the method disclosed herein further includes assessing serum creatinine (SCr) and serum cystatin C (SCysC) in serum samples of said subject at at least a first time point and a second time point on different days of said treatment period, and if the SCr or SCysC levels are within predetermined ranges, between said first and second time points, continuing administering the same predetermined daily dosage of voclosporin to said subject. In some examples, the predetermined range of SCr level is any one of about 0.5-2.0, 0.5-1.8, 0.5-1.6, 0.5-1.4, 0.5-1.21, 0.5-1, 0.84-2.0, 0.84-1.8, 0.84-1.6, 0.84-1.4, 0.84-1.21, or 0.5-1 mg/dL. In some examples, the predetermined range of SCr level is about any one of about <1.0, <1.1, <1.2, <1.21, <1.3, <1.4, <1.5, <1.6, <1.7, <1.8, <1.9, or <2.0 mg/dL. In some embodiments, the predetermined range of SCysC level is any one of about <0.5, <0.6, <0.7, <0.8, <0.9, <1.0, <1.1, <1.2, <1.3, <1.4, <1.5, <1.6, <1.7, <1.8, <1.9, or <2.0 mg/L. In some embodiments, the predetermined range of SCysC level is below 1 mg/L. In some examples, the predetermined range of SCr level is 0.84-1.21 mg/dL, and/or the predetermined range of SCysC level is below 1 mg/L.

In some embodiments, the method disclosed herein further includes assessing creatinine clearance (CrCl) and blood urea nitrogen (BUN) in serum samples of said subject at at least a first time point and a second time point on different days of said treatment period, and if the CrCl level is decreased below a first predetermined range and if BUN level is elevated above a second predetermined range, between said first and second time points, reducing the daily dosage by increment(s) of 7.9 mg BID or stopping the administering of voclosporin to said subject. In some embodiments, the method disclosed herein further includes assessing creatinine clearance (CrCl) and blood urea nitrogen (BUN) in serum samples of said subject at at least a first time point and a second time point on different days of said treatment period, and if the CrCl level is decreased below a first predetermined range or if BUN level is elevated above a second predetermined range, between said first and second time points, reducing the daily dosage by increment(s) of 7.9 mg BID or stopping the administering of voclosporin to said subject. In some embodiments, the method disclosed herein further includes assessing creatinine clearance (CrCl) and blood urea nitrogen (BUN) in serum samples of said subject at at least a first time point and a second time point on different days of said treatment period, and if the CrCl and BUN levels are within predetermined ranges, between said first and second time points, continuing administering the same predetermined daily dosage of voclosporin to said subject. In some embodiments, the method disclosed herein further includes assessing creatinine clearance (CrCl) and blood urea nitrogen (BUN) in serum samples of said subject at at least a first time point and a second time point on different days of said treatment period, and if the CrCl or BUN levels are within predetermined ranges, between said first and second time points, continuing administering the same predetermined daily dosage of voclosporin to said subject. In some embodiments, the first predetermined range of CrCl level is any one of about 120-170, 130-170, 130-160, 130-150, 135-170, 135-160, 135-150, 137-170, 137-137-160, 137-150 mL, or 140-145 mL/min in males, or any ranges there between. In some embodiments, the first predetermined range of CrCl level is any one of about 110-150, 120-150, 120-140, 120-135, 125-150, 125-140, 125-130, or 128-130 mL/min in females, or any ranges there between. In some embodiments the second predetermined range of BUN level is any one of about 5-30, 5-25, 6-22, 7-20, 8-18, 9-15, 10-12 mg/dL. In some embodiments, the first predetermined range of CrCl level is 137-150 mL/min in males and 128-130 mL/min in females, and the second predetermined range of BUN level is 7 to 20 mg/dL.

In some embodiments, the method disclosed herein further includes assessing renal vascular resistance and renal plasma flow (RPF) of said subject at at least a first time point and a second time point on different days of said treatment period, and if the renal vascular resistance and RPF are altered outside of predetermined values, between said first and second time points, reducing the daily dosage by increment(s) of 7.9 mg BID or stopping the administering of voclosporin to said subject. In some embodiments, the method disclosed herein further includes assessing renal vascular resistance and renal plasma flow (RPF) of said subject at at least a first time point and a second time point on different days of said treatment period, and if the renal vascular resistance or RPF are altered outside of predetermined values, between said first and second time points, reducing the daily dosage by increment(s) of 7.9 mg BID or stopping the administering of voclosporin to said subject. In some embodiments, the method disclosed herein further includes assessing renal vascular resistance and renal plasma flow (RPF) of said subject at at least a first time point and a second time point on different days of said treatment period, and if the renal vascular resistance and RPF remain within predetermined values, between said first and second time points, continuing administering the same predetermined daily dosage of voclosporin to said subject. In some embodiments, the method disclosed herein further includes assessing renal vascular resistance and renal plasma flow (RPF) of said subject at at least a first time point and a second time point on different days of said treatment period, and if the renal vascular resistance or RPF remain within predetermined values, between said first and second time points, continuing administering the same predetermined daily dosage of voclosporin to said subject. In some embodiments, the predetermined values of RPF is any one of about 500-800, 500-700, 510-690, 520-680, 530-670, 540-660, 550-650, 560-640, 570-630, 580-620, or 590-610 mL/min. In some embodiments, the predetermined values of RPF is any one of about 500-700, 520-700, 540-700, 560-700, 580-700, 600-700, 500-680, 500-660, 500-640, 500-620, or 500-620 mL/min. In some examples, the predetermined value of RPF is about 600 mL/min.

In some embodiments, the method disclosed herein further includes assessing albuminuria in morning and random urinary specimens of said subject at at least a first time point and a second time point on different days of said treatment period, and if the albuminuria is detected, between said first and second time points, reducing the daily dosage by increment(s) of 7.9 mg BID or stopping the administering of voclosporin to said subject. In some embodiments, the method disclosed herein further includes assessing albuminuria in morning and random urinary specimens of said subject at at least a first time point and a second time point on different days of said treatment period, and if the albuminuria is not detected, between said first and second time points, continuing administering the same predetermined daily dosage of voclosporin to said subject. Urine albumin can be analyzed and classified under the Kidney Disease: Improving Global Outcomes (KDIGO) stages of albuminuria and reported as milligrams of albumin divided by grams of creatinine. The stages include A1: Less than 30 mg/g, A2: 30 to 300 mg/g, and A3: Greater than 300 mg/g. In some embodiments, albuminuria is determined by the presence of albumin/creatinine ratios of greater than any one of: 5, 10, 15, 20, 25, 30, 35, 40, 50, 75, 100, 150, 200, 250, 300, 400 or 500 mg/g. In some embodiments, albuminuria is determined by the presence of albumin/creatinine ratios of greater than 30 mg/g.

In some embodiments, the method disclosed herein further includes assessing the level of one or more urinary electrolytes of said subject at at least a first time point and a second time point on different days of said treatment period, and if the level of the one or more urinary electrolyte is decreased or increased by more than a predetermined value, between said first and second time points, reducing the daily dosage by increment(s) of 7.9 mg BID or stopping the administering of voclosporin to said subject. In some embodiments, the method disclosed herein further includes assessing the level of one or more urinary electrolytes of said subject at at least a first time point and a second time point on different days of said treatment period, and if the level of the one or more urinary electrolyte is decreased or increased by less than the predetermined value, between said first and second time points, continuing administering the same predetermined daily dosage of voclosporin to said subject. In some of any embodiments, the urinary electrolyte is selected from one or more of magnesium, sodium and potassium.

In some of any embodiments, the urinary electrolyte is magnesium, and the predetermined value is about 1, 2, 3, 4, 5, 6, 7, 8, 9, 10, 11, 12, 13, 14, 15, 16, 17, 18, 19, 20, 25, 35, or 40 mg/dL. In some of any embodiments, the urinary electrolyte is magnesium, and the predetermined value is about 20 mg/dL. In some of any embodiments, the urinary electrolyte is sodium, and the predetermined value is about 5, 10, 15, 20, 25, 30, 35, 40, 45, 50, 60, 70, 80, 90 or 100 mmol/L. In some of any embodiments, the urinary electrolyte is sodium, and the predetermined value is about 50 mmol/L. In some of any embodiments, the urinary electrolyte is potassium, and the predetermined value is about 0.5, 1, 1.5, 2, 2.5, 3, 3.5, 4, 4.5, 5, 6, 7, 8, 9, 10, 11, 12, 13, 14, 15, 16, 17, 18, 19, 20, 21, 22, 23, 24 or 25 mmol/L. In some of any embodiments, the urinary electrolyte is potassium, and the predetermined value is about 10 mmol/L.

In some embodiments, the method disclosed herein further includes assessing the level of one or more lipids of said subject at at least a first time point and a second time point on different days of said treatment period, and if the level of the one or more lipid is outside of a predetermined range, at said second time point, reducing the daily dosage by increment(s) of 7.9 mg BID or stopping the administering of voclosporin to said subject. In some embodiments, the method disclosed herein further includes assessing the level of one or more lipids of said subject at at least a first time point and a second time point on different days of said treatment period, and if the level of the one or more lipid is within a predetermined range, at said second time point, continuing administering the same predetermined daily dosage of voclosporin to said subject. In some aspects, even if the subject exhibits a level of the one or more lipids that is outside of the predetermined range at the first time point, the subject receives administration of voclosporin at least until the second time point, at which time the level of the one or more lipids is within the predetermined range.

In some of any embodiments, the one or more lipid is selected from one or more of total cholesterol, high density lipoprotein (HDL) cholesterol, low density lipoprotein (LDL) cholesterol, and triglyceride. In some of any embodiments, the lipid is selected from one or more of total cholesterol, low density lipoprotein (LDL) cholesterol, and triglyceride.

In some of any embodiments, the lipid is total cholesterol, and the predetermined range is less than 170 mg/dL, 100-200 or 125-200 mg/dL. In some of any embodiments, the lipid is total cholesterol, and the predetermined range is 100-200 mg/dL. In some of any embodiments, the lipid is triglycerides, and the predetermined range is less than 150 mg/dL, or 50-150 mg/dL. In some of any embodiments, the lipid is triglycerides, and the predetermined range is 50-150 mg/dL. In some of any embodiments, the lipid is LDL, and the predetermined range is less than 100, 110, 120, or 130 mg/dL or 10-130, 30-130 or 50-130 mg/dL. In some of any embodiments, the lipid is LDL, and the predetermined range is 50-130 mg/dL. In some of any embodiments, the lipid is HDL, and the predetermined range is 40, 45 or 50 mg/dL or higher, or 35-60 mg/dL. In some of any embodiments, the lipid is LDL, and the predetermined range is 35-60 mg/dL.

In some embodiments according to any of the methods described herein, the predetermined daily dosage is 39.5 mg voclosporin BID or 31.6 mg voclosporin BID or 23.7 mg voclosporin BID or 15.8 mg voclosporin BID or 7.9 mg voclosporin BID. In some embodiments, the predetermined daily dosage is any one of about 7.9 to 23.7, 23.7 to 31.6, 31.6 to 39.5, or 39.5 to 79 mg voclosporin. In some embodiments, the predetermined daily dosage is about 2, 4, 6, 8, 10, 12, 14, 16, 18, 20, 24, 28, 32, 36, 40, 60, 80, 100 mg voclosporin, or any values there between.

In some embodiments, the first time point is any one of about 0.1, 0.5, 1, 2, 3, 4, 5, 6, 7, 8, 12, 16, 24, 36, or 48 hours preceding initiating said protocol. In any of the preceding embodiments, the first time point is immediately preceding initiating said protocol. In some embodiments, the second time point is any one of about 1, 2, 3, 4, 5, 10, 14, 15, 20, 25, 28, 40, 50 or more days subsequent to initiating said protocol, or any time periods there between. In some embodiments, the second time point is any one of about 1, 2, 3, 4, 5, 8 10, 12, 15, 16, 24, 25, 28, 30, 32, 35, 36, 40, 45, 50, 55, 60, 65, 70, 75, 80, 85, 90, 95, 100, 110, 120, 150 or more weeks subsequent to initiating said protocol, or any time periods there between.

In any of the preceding embodiments, the predetermined daily dosage of effective amounts of voclosporin is about any one of 5, 7.9, 10, 12, 15, 15.8, 20, 23.7, 25, 28, 30, 31.6, 35, 39.5, 40, 45, 50 mg voclosporin BID, or any amounts there between. In some embodiments, the predetermined daily dosage is 39.5 mg voclosporin BID or 31.6 mg voclosporin BID or 23.7 mg voclosporin BID or 15.8 mg voclosporin BID or 7.9 mg voclosporin BID.

In some embodiments according to any one of the methods described herein, wherein the subject has severe renal impairment at baseline, the predetermined daily dosage of effective amounts of voclosporin is about any one of 2, 5, 7.9, 10, 12, 15, 15.8, 20, 23.7, 25, 28, 31.6, 35 mg voclosporin BID, or any amounts there between. In some embodiments, wherein the subject has severe renal impairment at baseline, the predetermined daily dosage of effective amounts of voclosporin is about 15.8 mg voclosporin BID.

In some embodiments according to any one of the methods described herein, wherein the subject has moderate hepatic impairment, the predetermined daily dosage of effective amounts of voclosporin is about any one of 2, 5, 7.9, 10, 12, 15, 15.8, 20, 23.7, 25, 28, 31.6, 35 mg voclosporin BID, or any amounts there between. In some embodiments, wherein the subject has moderate hepatic impairment, the predetermined daily dosage of effective amounts of voclosporin is about 15.8 mg voclosporin BID.

In some embodiments according to any one of the methods described herein, wherein the subject is co-administered with one or more moderate CYP3A4 inhibitors, the predetermined daily dosage of effective amounts of voclosporin comprises a daily morning dosage of about any one of 2, 5, 7.9, 10, 12, 15, 15.8, 20, 23.7, 25, 28, 30, 31.6, 35 mg voclosporin, or any amounts there between; and an afternoon dosage of about any one of 1, 2, 5, 7.9, 10, 12, 15, 15.8, 20, or any amounts there between. In some embodiments, wherein the subject is co-administered with one or more moderate CYP3A4 inhibitors, the predetermined daily dosage of effective amounts of voclosporin comprises a daily morning dosage of about 15.8 mg voclosporin, and a daily afternoon dosage of about 7.9 mg voclosporin. In some embodiments, the moderate CYP3A4 inhibitors comprises one or more of verapamil, fluconazole, diltiazem.

In any of the preceding embodiments, the method further includes evaluating said subject for renal function at a time point after the end of said treatment period by assessing eGFR. In some embodiments, the method further includes evaluating said subject for efficacy by assessing protein/creatinine ratio (UPCR) at a time point after the end of said treatment period.

In any of the preceding embodiments, the method further includes administering to said subject an effective amount of mycophenolate mofetil (MMF). In some embodiments, the method further includes administering to said subject an effective amount of a corticosteroid.

In some embodiments, the methods and uses provided herein can be used for a prolonged or an extended treatment regimen, and/or to reduce chronic calcineurin inhibitor nephrotoxicity. In some embodiments, the treatment period is at least 55 weeks. In some embodiments, the treatment period is at least 100 weeks. In some of any embodiments, the treatment period is at least 150 weeks. In some aspects, the treatment period is at least one year, two years or three years.

In any of the preceding embodiments, the method further includes determining the eGFR of the subject at a third time point and if the eGFR is determined at said third time point to differ from the eGFR determined at said first time point by less than said target %, resuming administering said predetermined daily dosage of voclosporin. In some examples, the target % is 20-45%. In some examples, the target % is approximately 30%.

In some embodiments, disclosed herein is a method to reduce chronic calcineurin inhibitor nephrotoxicity in treatment of a proteinuric kidney disease or associated with a transplant, in which the method includes administering to a subject diagnosed with said disease predetermined daily dosages of effective amounts of voclosporin over a projected treatment period to an end point, and further includes measuring urinary protein creatinine ratio (UPCR) of said subject at a first time point prior to said treatment period and a second time point occurring prior to the end point but after the start of the treatment period and determining any reduction of said UPCR, between said first and second time points, and, if the UPCR of said subject fails to show a reduction of at least a predetermined amount at said second time point, discontinuing administering voclosporin to the subject and continuing said administering if said predetermined amount of reduction is shown.

In some embodiments, the method described herein further comprises measuring the concentration of C3/C4 in the blood of said subject at said first and second time points and determining whether the concentration of C3/C4 is normalized at said second time point and, if said normalization is found, reinstating or continuing administering voclosporin to the subject and if normalization has not occurred maintaining said discontinuing. In some aspects, the method further includes administering to said subject an effective amount of mycophenolate mofetil (MMF). In some examples, the method further includes administering to the subject an effective amount of a corticosteroid. In some embodiments, the predetermined daily dosage is 39.5 mg voclosporin BID or 31.6 mg voclosporin BID or 23.7 mg voclosporin BID or 15.8 mg voclosporin BID or 7.9 mg voclosporin BID.

In some embodiments, disclosed herein is a method to treat a proteinuric kidney disease which includes administering to a subject diagnosed with lupus nephritis a predetermined daily dosage of effective amounts of voclosporin over a projected treatment period of at least 8 weeks, wherein said effective amount is 15.8 mg BID or 7.9 mg voclosporin BID.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### EXAMPLES

The following example is included for illustrative purposes only and is not intended to limit the scope of the present disclosure.

### Example 1: 48 Week Study of LN Treatment

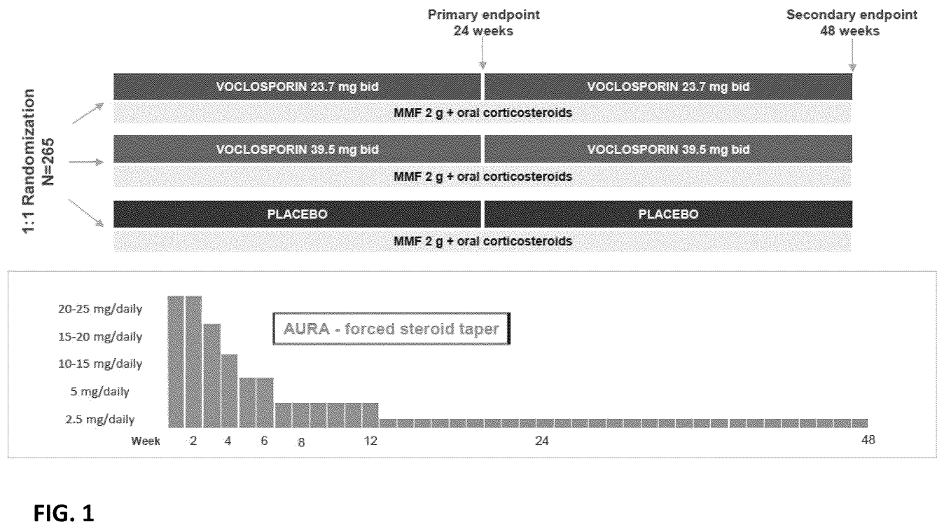

The subjects enrolled in the study were divided into three groups, 88 subjects were in a control group who were administered 2 g MMF daily as well as oral corticosteroids—i.e., prednisone in a tapering dosage shown graphically in FIG. 1—beginning at 20-25 mg daily reduced gradually after the 12th week to 2.5 mg daily. 89 subjects in the low dosage group received this background treatment, but in addition were administered three capsules containing 7.9 mg (i.e. 23.7 mg) of voclosporin each twice daily. The voclosporin used in this study comprised greater than 90% E isomer. A third group which was comprised of 88 subjects received a similar background treatment but in addition were dosed with five 7.9 mg capsules i.e. 39.5 mg twice daily. The study was conducted over a period of 48 weeks and safety was evaluated at 24 weeks.

Subjects were screened prior to admission to the study by (a) determining that the urine protein creatinine ratio (UPCR) as >1.5 mg/mg as measured by first morning void, and (b) that the eGFR as measured by Chronic Kidney Disease Epidemiology Collaboration equation (CKD-EP1) of >45 ml/min/1.73 m². Subjects were assessed after 24 weeks and 48 weeks as well as a subsequent evaluation at 50 weeks.

The low dosage administration achieved better results than administration of voclosporin at higher dosages. Briefly, 32.6% of low dosage patients showed CR at 24 weeks compared to 19.3% of controls and 70% showed PR compared to 49% of controls.

CR in this example is a composite end-point which includes efficacy, safety and low-dose steroids: UPCR≤0.5 mg/mg (confirmed); eGFR>60 ml/min/1.73 m² or within 20% of baseline; steroids ≤10 mg/day; no administration of rescue medication.

PR is a composite end-point that includes safety and efficacy: UPCR reduction of 50% from baseline and no use of rescue medication.

To determine the efficacy of the pharmacodynamics protocol wherein dosage is reduced or stopped according to the presence or absence of indicators of the decrease in eGFR experienced as a side effect, these three groups of patients were assessed after 24 weeks and 48 weeks of treatment in consideration of whether treatment was altered according to the invention protocol. In all three groups, the patients were evaluated according to the criteria set forth in the exemplary protocol above—i.e., wherein the eGFR of each patient was measured immediately prior to administering the first dose of voclosporin and at a second time point at least a day later and
(i) if the eGFR of said subject decreases by >30% to a value of below 60 mL/min/1.73 m², between said first and second time points, stopping the administering of voclosporin to said subject;
(ii) if the eGFR of said subject decreases by between 20% to 30% to a value of below 60 ml/min/1.73 m², between said first and second time points, administering a reduced dosage of voclosporin to said subject;
(iii) if the eGFR of said subject decreases by ≤20%, between said first and second time points, continuing administering the same predetermined daily dosage of voclosporin to said subject.

The results are shown in Tables 2 and 3 below. Table 2 shows percentages with complete remission (CR) or partial remission (PR) after 24 weeks and Table 3 shows these values after 48 weeks for patients who had no dose reduction and those who did have dose reduction.

TABLE 2
Patients with a No Dose Reductions (24 weeks):

| | | Patients with | Patients | Patients |
|---|---|---|---|---|
| | Patient | no Dose | with CR at | with PR at |
| | number | Reduction | 24 weeks | 24 weeks |
| Group | (n) | n(%) | n(%) | n(%) |
| Placebo | 88 | 77 (87.5) | 14 (18.2) | 36 (46.8) |
| Low Dose | 89 | 50 (56.2) | 15 (30.0) | 34 (68.0) |
| High Dose | 88 | 41 (46.6) | 11 (26.8) | 24 (58.5) |

Patients with Dose Reductions (pharmacodynamically dosed):

| | | Patients with | Patients | | Patients | |
|---|---|---|---|---|---|---|
| | Patient | Dose | with CR at | | with PR at | |
| | number | Reduction | 24 weeks | | 24 weeks | |
| Group | (n) | n(%) | n(%) | | n(%) | |
| Placebo | 88 | 11 (12.5) | 3 | (27.3) | 7 | (63.6) |
| Low Dose | 89 | 39 (43.8) | 14 | (35.9) | 28 | (71.8) |
| High Dose | 88 | 47 (53.4) | 13 | (27.7) | 34 | (72.3) |

TABLE 3
Patients with No Dose Reductions (48 weeks):

| | | Patients with | Patients | Patients |
|---|---|---|---|---|
| | Patient | no Dose | with CR at | with PR at |
| | number | Reduction | 48 weeks | 48 weeks |
| Group | (n) | n(%) | n(%) | n(%) |
| Placebo | 88 | 74 (84.1) | 18 (24.3) | 38 (51.4) |
| Low Dose | 89 | 43 (48.3) | 20 (46.5) | 26 (60.5) |
| High Dose | 88 | 35 (39.8) | 11 (31.4) | 22 (62.9) |

Patients with Dose Reductions (pharmacodynamically dosed):

| | | Patients with | Patients | | Patients | |
|---|---|---|---|---|---|---|
| | Patient | Dose | with CR at | | with PR at | |
| | number | Reduction | 48 weeks | | 48 weeks | |
| Group | (n) | n(%) | n(%) | | n(%) | |
| Placebo | 88 | 14 (15.9) | 3 | (21.4) | 4 | (28.6) |
| Low Dose | 89 | 46 (51.7) | 24 | (52.2) | 35 | (76.1) |
| High Dose | 88 | 53 (60.2) | 24 | (45.3) | 41 | (77.4) |

In this study, CR was defined as a composite of UPCR≤0.5 mg/mg; eGFR>60 mL/min/1.73 m² or within 20% of baseline, steroids at ≤10 mg/day and no administration of rescue medication. PR is defined as UPCR reduction of 50% from baseline and no use of rescue medication.

As shown in Table 2, after 24 weeks, 12.5% of patients on placebo, 43.8% of patients on low dose and 53.4% of patients on high dose voclosporin underwent dose reduction during the treatment. The percentage of patients with complete response was not affected in either dosage groups by the pharmacodynamic dosage and the percentage with partial response was also roughly the same, although with the high dose group, the percentage with partial reduction improved. Table 3 shows similar results at 48 weeks, although a higher percentage of patients were subjected to dose reduction. Again, no drastic effect on the overall response was exhibited.

### Example 2: Low Dosage Protocol

In the course of clinical studies similar to those in Example 1, it was observed that a substantial portion of subjects showed substantial remission at a dosage reduced almost immediately to 15.8 mg voclosporin administered twice daily (BID). Accordingly, applicants have analyzed these data and have concluded that a dosage protocol providing 15.8 mg or 7.9 mg voclosporin BID is effective with or without the pharmacodynamics aspects of the protocol.

As the capsules contain 7.9 mg voclosporin, 1 cap represents 7.9 mg voclosporin, 2 caps represent 15.8 mg voclosporin and 3 caps represent 23.7 mg voclosporin, etc. Substantial numbers of subjects showed complete or partial remission even when the dosage was lowered to 7.9 mg voclosporin BID quite early in the treatment and similar results were obtained for administration of 15.8 mg BID.

### Example 3: Low Dose Corticosteroid

Applicants have also found that the dosage of corticosteroid can effectively be reduced as compared to “standard of care” as shown in Tables 4 and 5, and can be reduced further to 4 mg per day or less.

TABLE 4
Standard of Care Dosing Schedule for IV methylprednisolone
and daily oral prednisone:

| | Patients <45 kg | Patients ≥45 kg |
|---|---|---|
| Time | (daily dosage) | (daily dosage) |
| Days 1-3 | 0.5 g IV methylprednisolone | 1 g IV methylprednisolone |
| Days 3-112 | 1 mg/kg tapered down | 1 mg/kg (maximum 80 mg) |
| | | tapered down |

TABLE 5
Lowered Dosing Schedule for IV methylprednisolone
and daily oral prednisone:

| | Patients <45 kg | Patients ≥45 kg |
|---|---|---|
| Time | (daily dosage) | (daily dosage) |
| Days 1-2 | 0.25 g IV methylprednisolone | 0.5 g IV methylprednisolone |
| Days 3-13 | 20 mg oral prednisone | 25 mg oral prednisone |
| Week 3 | 15 mg oral prednisone | 20 mg oral prednisone |
| Week 4 | 10 mg oral prednisone | 15 mg oral prednisone |
| Week 6 | 10 mg oral prednisone | 10 mg oral prednisone |
| Week 8 | 5 mg oral prednisone | 5 mg oral prednisone |
| Week 12 | 5 mg oral prednisone | 5 mg oral prednisone |
| Week 16 | 2.5 mg oral prednisone | 2.5 mg oral prednisone |

### Example 4: Post-55 Week Follow-Up Study of Ln Treatment

The subjects enrolled in the study, as described in Example 1, are assessed after 55 weeks of treatment, to assess whether to alter the treatment according to the protocol as described herein.

In all three groups, the subjects are evaluated according to the criteria as set forth in the exemplary protocol described in the application above—i.e., wherein the eGFR of each patient is measured immediately prior to administering the first dose of voclosporin and at a second time point at least a day later, and:
(i) if the eGFR of said subject decreases by ≥30% to a value of below 60 mL/min/1.73 m², between said first and second time points, stopping the administering of voclosporin or reducing dosage thereof to said subject;
(ii) if the eGFR of said subject decreases by between 20% to 30% to a value of below 60 ml/min/1.73 m², between said first and second time points, administering a reduced dosage of voclosporin to said subject;
(iii) if the eGFR of said subject decreases by ≤20%, between said first and second time points, continuing administering the same predetermined daily dosage of voclosporin to said subject.

### Example 5: Assessment of Interstitial Fibrosis and Tubular Atrophy

Renal biopsies are collected at various time points during the course of the study as described in Example 4. All biopsies are assessed using light microscopy, immunofluorescent microscopy and electron microscopy. The collected renal biopsies are assessed by hematoxylin and eosin staining (H&E), periodic acid-Schiff (PAS), silver and trichrome stains and collagen III immunohistochemistry and are graded semiquantitatively using the following categories: Grade 0: ≤5%; Grade 1: 5-25%; Grade 2: 25-≤50%; Grade 3: >50%. The categories represent the percentage of non-scarred cortical area involved by mononuclear cell infiltrate and are analogous to the Banff interstitial inflammation scores for kidney allograft rejection. The trichrome stain is used to aid in the identification of fibrosis. Interstitial fibrosis is identified as the accumulation of collagen and related molecules in the interstitium Tubular atrophy are identified by small tubules with cells with pale cytoplasm or dilated, thin tubules.

If the renal biopsies reveal the presence of interstitial fibrosis and tubular atrophy between the first and second time points described in Example 4, administration of voclosporin is stopped or reduced to said subject. If the renal biopsies do not reveal the presence of interstitial fibrosis and tubular atrophy, between said first and second time points, administration of voclosporin is continued at the same predetermined daily dosage to said subject.

### Example 6: Assessment of Medial Arteriolar Hyalinosis

Renal biopsies, obtained as described in Example 5, are further assessed for medial arteriolar hyalinosis. The presence of nodular hyaline deposits in the media of afferent arterioles is identified by the replacement of necrotic smooth muscle cells with focal or circular lumpy protein (hyaline) deposits at the periphery of the wall of afferent arterioles. Further evaluation includes assessment of the narrowing of the vascular lumen, which is negatively associated with positive renal outcome.

If the renal biopsies reveal the presence of medial arteriolar hyalinosis between the first and second time points described in Example 4, administration of voclosporin is stopped or reduced to said subject. If the renal biopsies do not reveal the presence of medial arteriolar hyalinosis, between said first and second time points, administration of voclosporin is continued at the same predetermined daily dosage to said subject.

### Example 7: Assessment of Glomerular Capsular Fibrosis, Global Glomerulosclerosis and Focal Segmental Glomerulosclerosis (FSGS)

Renal biopsies, obtained as described in Example 5, are further assessed for glomerular injury. Global glomerulosclerosis is identified by the presence of arteriolar hyalinosis, arteriolopathy, secondary glomerular ischemia, and/or atubular glomeruli. The presence of atubular glomeruli is characterized by the presence of glomeruli that are disconnected from the proximal tubule. Atubular glomeruli are also identified by the presence of periglomerular fibrosis (capsular fibrosis), or by the presence of severely contracted glomeruli within an enlarged glomerular cyst. Focal segmental glomerulosclerosis lesions are identified by the presence of sclerosis in parts of at least one glomerulus, and are identified as approximately 12.5 percent of the total glomerular volume.

Global and segmental glomerulosclerosis is scored from 0 to 3, tubular atrophy from 0 to 3, interstitial fibrosis from 0 to 3 and arteriosclerosis from 0 to 1. The scores are then added (total renal chronicity score) to grade the overall severity of the chronic lesions into minimal (0-1 total score), mild (2-4 total score), moderate (5-7 total score) and severe (≥8 total score).

If the renal biopsies reveal the presence of glomerular injury between the first and second time points described in Example 4, administration of voclosporin is stopped or reduced to said subject. If the renal biopsies do not show the presence of glomerular injury, between said first and second time points, administration of voclosporin is continued at the same predetermined daily dosage to said subject.

### Example 8: Assessment of Juxtaglomerular Apparatus Hyperplasia

Renal biopsies, obtained as described in Example 5, are further assessed for juxtaglomerular apparatus (JGA) hyperplasia. Renal biopsies are stained with Masson and immunoperoxidase stains, and immunohistochemical analysis is performed. Samples are further analyzed by electron microscopy. JGA hyperplasia is assessed as enlargement of the juxtaglomerular apparatus components, including the vascular components (portions of afferent and efferent arterioles), the mesangial cell components (modified extraglomerular and intraglomerular smooth muscle cells), and the tubular components (the macula densa), as well as the presence of intracellular renin granules.

If the renal biopsies reveal the presence of JGA hyperplasia between the first and second time points described in Example 4, administration of voclosporin is stopped or reduced to said subject. If the renal biopsies do not reveal the presence of JGA hyperplasia, between said first and second time points, administration of voclosporin is continued at the same predetermined daily dosage to said subject.

### Example 9: Assessment of Tubular Microcalcifications

Renal biopsies, obtained as described in Example 5, are further assessed for tubular microcalcifications. Histological slides of undecalcified formalin (4%) fixed, paraffin-embedded biopsies are analyzed with haematoxylin and eosin staining (H&E) and periodic acid Schiff (PAS) stains, as well as von Kossa and Alizarin stains for visualization of calcium phosphate depositions. Electron microscopy is used to further identify deposition of calcium in renal tubules. The density of microcalcifications is evaluated as the number of calcification foci per square millimeter of biopsy, and calculated by dividing the total number of calcification foci found in the biopsy by the total area of the biopsy measured on the slide (foci per square millimeter).

If the renal biopsies reveal the presence of tubular microcalcifications between the first and second time points described in Example 4, administration of voclosporin is stopped or reduced to said subject. If the renal biopsies do not reveal the presence of tubular microcalcifications, between said first and second time points, administration of voclosporin is continued at the same predetermined daily dosage to said subject.

### Example 10: Assessment of P-Glycoprotein Expression Creatinine Clearance

Renal biopsies, obtained from subjects generally as described in Example 5, are assessed for P-glycoprotein expression. P-glycoprotein (P-gp) expression is analyzed by the staining of renal biopsies using anti-P-glycoprotein antibodies. P-glycoprotein expression in glomeruli and tubular epithelial cells is scored semi-quantitatively according to the intensity and distribution. The classification includes: normal=0 point, P-gp expression loss in <10% of tubules placed in the cortical area; mild loss=1 point, P-gp expression loss in 10% to 24% of tubuli in the cortical area; moderate loss=2 points, P-gp expression loss in 25% to 50% of tubuli in the cortical area; severe loss=3 points, P-gp expression loss in >50% of tubuli in the cortical area. A decrease in expression of P-glycoprotein is suggestive of renal pathology.

If the renal biopsies reveal the loss of expression of P-gp outside of a predetermined range, between the first and second time points described in Example 4, administration of voclosporin is stopped or reduced to said subject. If the renal biopsies do not reveal a loss of expression of P-gp outside of a predetermined range, between said first and second time points, administration of voclosporin is continued at the same predetermined daily dosage to said subject.

### Example 11: Assessment of Calcineurin Inhibitor Nephrotoxicity and Banff Score

To further determine the efficacy of the pharmacodynamics protocol described above, renal biopsies, obtained as described in Example 5, are further assessed for Calcineurin Inhibitor (CNI) Nephrotoxicity and Banff Scores. For grading the severity of chronic changes, all biopsies are scored in a semiquantitative manner. The CNI Nephrotoxicity score is evaluated based on six parameters that are graded on a scale of 0 to 3, resulting in a possible total score of 18. The six parameters include: (1) isometric tubular vacuolization (tv), (2) peripheral or medial arteriolar hyaline (ah), (3) striped interstitial fibrosis, (4) ischemic collapse of glomeruli, (5) juxtaglomerular apparatus hyperplasia, and (6) tubular dystrophic calcifications. To determine the Banff Chronicity Score (BChS), renal biopsies are analyzed for transplant glomerulopathy (cg), tubular atrophy (ct), interstitial fibrosis (ci), and chronic vascular changes (cv), and are graded on a scale of 0 to 3. The severity of renal pathology is categorized into mild, moderate, and severe.

If the renal biopsies reveal a CNI Nephrotoxicity score, or a Banff Chronicity Score (BChS), outside of a predetermined range, between the first and second time points described in Example 4, administration of Voclosporin is stopped or reduced to said subject. If the renal biopsies do not reveal a CNI Nephrotoxicity score, or a Banff Chronicity Score (BChS), outside of a predetermined range, between said first and second time points, administration of Voclosporin is continued at the same predetermined daily dosage to said subject.

### Example 12: Assessment of Activity and Chronicity Indexes

Renal biopsies, obtained as described in Example 5, are further assessed using the National Institutes of Health Activity Index (NIH-AI), the National Institutes of Health Chronicity Index (NIH-CI), and the Tubulointerstitial Activity Index (TIAI). Biopsy slides were stained for hematoxylin and eosin (H&E), trichrome, and immunofluorescence with C3, C4, C5, C1q, IgG, IgM, and IgA. Electron microscopy pictures are collected to facilitate the interpretation.

The indices comprise scoring the percentage of glomeruli that display each feature described below in the biopsy on a scale of 0 to 3 (a score of 0=not present, 1=<25% glomeruli, 2=25-50% glomeruli, and 3 indicating >50% glomeruli).

For the NIH AI, indicators of disease activity are evaluated, which include endocapillary hypercellularity, neutrophils or karyorrhexis within glomerular capillary loops, fibrinoid necrosis, hyaline deposits, cellular or fibrocellular crescents, and interstitial inflammation. Crescents and fibrinoid necrosis are weighted twice as they have a worse impact on prognosis. The score range is 0-24, with 0 considered inactive. A high NIH-AI value (>6) is considered a significant change.

Indicators of NIH-CI include the total percentage of global glomerulosclerosis, fibrous crescents, tubular atrophy, and interstitial fibrosis. The NIH-CI score range is 0-12, with 0 representing no chronicity. A high NIH-CI value (>3) is correlated with progression to renal failure.

For the TIAI, tubular cell pyknosis, nuclear activation, necrosis, flattening, macrophages in the tubular lumens, epithelial cells in the tubular lumens, and interstitial inflammation are evaluated. The TIAI score range is 0-21, with 0 representing no interstitial activity.

Statistical analysis is performed to determine the frequencies for categorical variables. P-values≤0.05 are considered statistically significant.

If the renal biopsies reveal an NIH-AI, a NIH-CI, or a TIAI, outside of a predetermined range, between the first and second time points described in Example 4, administration of voclosporin is stopped or reduced to said subject. If the renal biopsies do not reveal an NIH-AI, an NIH-CI, or a TIAI, outside of a predetermined range, between said first and second time points, administration of voclosporin is continued at the same predetermined daily dosage to said subject.

### Example 13: Assessment of Distal Tubular Acidosis (Hyperchloremic Metabolic Acidosis)

Urinary specimens are collected at various time points during the course of the study, substantially as described in Example 4, and assessed using an NH4+ loading test involving administration of ammonium chloride (NH₄Cl) in a quantity sufficient to acidify urine. Urinary pH is calculated using the urine anion gap (UAG) as a surrogate marker for NH4⁺ secretion. The UAG is derived by subtracting urine chloride (Cl⁻) from sodium (Na⁺) and potassium (K⁺) ions present in the urine.

The absence of a decrease in urinary pH is detected by an inappropriately positive UAG, as characterized by a range above 20-90 mEq/L. A positive UAG result is used as an indicator of distal tubular acidosis.

If a positive UAG is detected between the first and second time points described in Example 4, administration of voclosporin is stopped or reduced to said subject. If a negative UAG is detected, between said first and second time points, administration of voclosporin is continued at the same predetermined daily dosage to said subject.

### Example 14: Assessment of Ion Homeostasis

Serum and urine samples are obtained from subjects at different time points throughout the study as described in Example 4, and are assessed for hyperkalemia, hypomagnesemia, magnesium wasting and hyperuricemia. Hyperkalemia is evaluated from collected serum samples and classified into mild (5.1-<6 mmol/l), moderate (6-<7 mmol/l) and severe (≥7 mmol/l). Low serum magnesium (hypomagnesemia) is determined as serum magnesium levels below the standard reference range, or less than 1.4-1.8 mg/dL. Urine analysis exhibiting magnesium excretion of more than 2 mEq (1 mmol or 24 mg) is classified as indicative of renal magnesium wasting. Elevated uric acid (hyperuricemia) is evaluated from collected serum samples, and are classified as values above the standard upper limit of 6.0-7.0 mg/dL.

If hyperkalemia, hypomagnesemia, magnesium wasting or hyperuricemia are detected between the first and second time points described in Example 4, administration of voclosporin is stopped or reduced to said subject. If hyperkalemia, hypomagnesemia, magnesium wasting or hyperuricemia are not detected, between said first and second time points, administration of voclosporin is continued at the same predetermined daily dosage to said subject.

### Example 15: Assessment of Serum Creatinine and Cystatin C

Serum samples are collected from the subjects enrolled in the study as described in Example 4. Serum creatinine (SCr) is estimated by an enzymatic method and the Jaffe method. Serum cystatin C (SCysC) is estimated by particle-enhanced nephelometric immunoassay (PENIA) and particle-enhanced turbidimetric immunoassay (PETIA). Elevated SCr levels are determined as values above a normal range of 0.84-1.21 mg/dL (74.3-107 mmol/L). Elevated SCysC levels are determined as values above 1 mg/L.

If elevated SCr or SCysC levels are detected between the first and second time points described in Example 4, administration of voclosporin is stopped or reduced to said subject. If elevated SCr or SCysC levels are not detected, between said first and second time points, administration of voclosporin is continued at the same predetermined daily dosage to said subject.

### Example 16: Assessment of Creatinine Clearance and Blood Urea Nitrogen (BUN)

The subjects enrolled in the study as described in Example 4 are further assessed for creatinine clearance and blood urea nitrogen. Subject demographics, including height and weight, are recorded, and serum creatinine (SCr) is determined as described in Example 15. A timed urine sample is collected recording time, volume and creatinine concentration.

Creatinine clearance (CrCl) is calculated using the Cockcroft-Gault equation CrCl=((140−Age)*(weight in kg)/72*SCr) with a correction of *0.85 in females. Creatinine clearance (CrCl) is also calculated using the Dubois and Dubois formula CrCl=(Urinary Creatinine*urinary volume)/(Serum creatinine*time in minutes). Decreased values outside the standard upper range of creatinine clearance of 137-150 mL/min in males and 128-130 mL/min in females are suggestive of renal pathology.

Serum samples are collected and analyzed for blood urea nitrogen (BUN) levels. BUN is determined quantitatively utilizing standard instrumentation. An increase in BUN above the standard range of 7 to 20 mg/dL (2.5 to 7.1 mmol/L) is suggestive of renal pathology.

If decreased CrCl values or elevated BUN levels are detected between the first and second time points described in Example 4, administration of voclosporin is stopped or reduced to said subject. If decreased CrCl values or elevated BUN levels are not detected, between said first and second time points, administration of voclosporin is continued at the same predetermined daily dosage to said subject.

### Example 17: Assessment of Renal Vascular Resistance and Renal Plasma Flow

The subjects enrolled in the study as described in Example 4 are further assessed for renal vascular resistance and renal plasma flow (RPF). Renal vascular resistance is calculated as the renal blood flow divided by mean arterial pressure. Renal blood flow is measured by a miniaturized pulsed Doppler probe fixed on a renal artery. Mean arterial pressure is determined by measurement of blood pressure utilizing an oscillometric device, and followed by calculation using the following formula: (systolic blood pressure+2(diastolic blood pressure))/3.

To assess renal plasma flow, serum and urine samples are collected generally as described in the Examples above, and analyzed for Para-aminohippuric acid (PAH). Urine flow rate and volume are determined in cc/min. RPF is calculated as (PAH in urine*urine flow rate (cc/min))/PAH in plasma. Values outside the standard range of 600 ml/min are suggestive of renal pathology.

If alterations in renal vascular resistance or RPF are detected between the first and second time points described in Example 4, administration of voclosporin is stopped or reduced to said subject. If alterations in renal vascular resistance or RPF are not detected, between said first and second time points, administration of voclosporin is continued at the same predetermined daily dosage to said subject.

### Example 18: Assessment of Albuminuria

Urine albumin is measured in 24-hour urine collections or early morning/random specimens as an albumin/creatinine ratio in the study as described in Example 4. Samples are analyzed and classified under the Kidney Disease: Improving Global Outcomes (KDIGO) stages of albuminuria and reported as milligrams of albumin divided by grams of creatinine. The stages include A1: Less than 30 mg/g, A2: 30 to 300 mg/g, and A3: Greater than 300 mg/g.

The presence of albuminuria on two occasions with the exclusion of a urinary tract infection is suggestive of glomerular dysfunction. The presence of albuminuria for three or more months is suggestive of chronic kidney disease.

If albuminuria is detected between the first and second time points described in Example 4, administration of voclosporin is stopped or reduced to said subject. If albuminuria is not detected, between said first and second time points, administration of voclosporin is continued at the same predetermined daily dosage to said subject.

### Example 19: Assessment of Electrolyte Profiles in Lupus Nephritis Patients Treated with Voclosporin

Electrolyte imbalances, modulating within the kidney, have been reported in connection with use of calcineurin inhibitors (CNIs) in solid organ transplantation (Gratreak et al., Physiological reports. 2020; 8(1):e14316-e; Deray et al., Ann Intern Med. 1992; 117(7):578-83). A clinical finding of kidney toxicity from use of CNIs can be supported by, for example, evaluating changes in urinary and/or serum electrolyte concentrations over time. The effect of the CNI voclosporin in combination with immunosuppressive therapy on the urinary electrolytes of subjects with lupus nephritis were investigated.

A double-blind randomized controlled trial was conducted in subjects with active lupus nephritis to evaluate the efficacy and safety of oral voclosporin (23.7 mg twice daily) in comparison to a placebo. MMF (target dose 1 g twice daily) and low-dose oral steroids (rapidly tapered to 2.5 mg daily at week 16) were also administered to subjects in both the voclosporin and placebo groups.

Patients with lupus nephritis treated with voclosporin in combination with MMF and low-dose steroids achieved significantly higher complete renal response rates compared to patients treated with only MMF and low-dose steroids (at one year: 40.8% vs 22.5%; OR 2.65; p<0.0001).

Twenty-four-hour urine samples from 60 patients in each treatment arm were selected for urinary analysis of electrolytes. Samples were included from patients who responded to treatment at one year with at least 50% reduction in urine protein creatinine ratio (UPCR) from baseline (treatment responders), patients who had not (non-responders), and patients who had at least 30% reduction from baseline in estimated glomerular filtration rate (eGFR) during the study. The mean change from baseline to one year in magnesium, potassium and sodium were presented as 24-hour urinary excretion concentrations.

Mean changes from baseline in urinary electrolyte excretion concentrations were small for magnesium (<13.4 mg/dL) and potassium (<6.4 mmol/L), with no significant differences between treatment arms for any of the subgroups. Sodium excretion concentrations decreased from baseline in all subgroups (<30.5 mmol/L) except for responders in the control arm (increase of 14.0 mmol/L) at one year (Table 6).

TABLE 6*
Analysis of electrolyte urinary excretion concentrations

| Responders | | | Non-responders | | | eGFR decrease | | |
|---|---|---|---|---|---|---|---|---|
| Voclosporin | Control | P-value | Voclosporin | Control | P-value | Voclosporin | Control | P-value |

Change from baseline, mean ± SEM

| | | | | | | | | | |
|---|---|---|---|---|---|---|---|---|---|
| Magnesium | n = 20 | n = 26 | 0.5472 | n = 25 | n = 19 | 0.4295 | n = 28 | n = 20 | 0.0745 |
| | 4.7 ± 42 | 1.3 ± 28 | | −3.9 ± 6.44 | −1.0 ± 7.40 | | −13.4 ± 6.06 | 0.7 ± 7.34 | |
| Sodium | n = 28 | n = 30 | 0.0352 | n = 32 | n = 30 | 0.3728 | n = 28 | n = 20 | 0.4615 |
| | −27.6 ± 109 | 14.0 ± 71.8 | | −23.0 ± 12.69 | −6.3 ± 13.10 | | −30.5 ± 11.92 | −16.0 ± 14.11 | |
| Potassium | n = 28 | n = 30 | 0.2914 | n = 32 | n = 30 | 0.8696 | n = 28 | n = 20 | 0.7864 |
| | −6.38 ± 24.0 | 3.13 ± 21.2 | | −2.48 ± 4.806 | −3.14 ± 4.963 | | −4.58 ± 4.648 | −1.53 ± 5.501 | |

*Subgroup analysis of 60 patients is presented. Mean ± SEM change from baseline to one year of treatment in the trial was analyzed with 24-hour urinary excretion concentrations. Responders were defined as having ≥50% reduction from baseline in urine protein creatinine ratio at one year. Patients who had ≥30% reduction from baseline in estimated glomerular filtration rate (eGFR) during the study were included in eGFR decrease cohort.

Mean serum electrolyte concentrations of patients treated with voclosporin were within normal ranges (Rovin et al., Lancet. 2021 May 29; 397(10289):2070-2080). At the studied dose of voclosporin, changes from baseline in urinary electrolyte excretion concentrations of magnesium, potassium, and sodium were not clinically meaningful in either treatment arm regardless of response to treatment.

The results of the study, together with the reported normal electrolyte concentrations in serum, confirms that voclosporin does not have substantial impact on the mean concentrations of electrolytes. The results further support the safety and efficacy of voclosporin for the treatment of patients with lupus nephritis, particularly for patients who may be at elevated risk for CNI nephrotoxicity.

### Example 20: Localization of Cyclosporine A, Voclosporin, and Metabolites in Mouse Kidney Tissues by Matrix-Assisted Laser Desorption/Ionization-Mass Spectrometric Imaging (MALDI-MSI)

Although cyclosporine A and voclosporin are structurally similar, the two compounds display different metabolic stability and distribution, which may contribute to distinct efficacy and safety profiles. Certain metabolites of cyclosporine A have been correlated with nephrotoxicity (Wu and Kuca, Current Drug Metabolism 2019; 20(2):84-90(7)). The localization and distribution of cyclosporine A, voclosporin, and their metabolites in mouse kidneys were assessed following IV administration, using matrix assisted laser desorption/ionization mass spectrometry imaging (MALDI-MSI). MALDI-MSI provides high sensitivity multiplex analysis of the spatial orientation of molecules in a biological tissue sample without the need for labeling.

Major metabolic pathways of cyclosporine A in humans include hydroxylation and N-demethylation. These processes yield metabolites of relatively less toxicity than the parent compound, but metabolism in the kidney is not as efficient as in the liver. Metabolites of cyclosporine A include AM1, AM1c, AM4N, AM9, AM19, and AM1c9. Some metabolites, such as AM19 and AM1c9, are correlated with nephrotoxicity in subjects treated with cyclosporine A (Wu and Kuca, Current Drug Metabolism 2019; 20(2):84-90(7)).

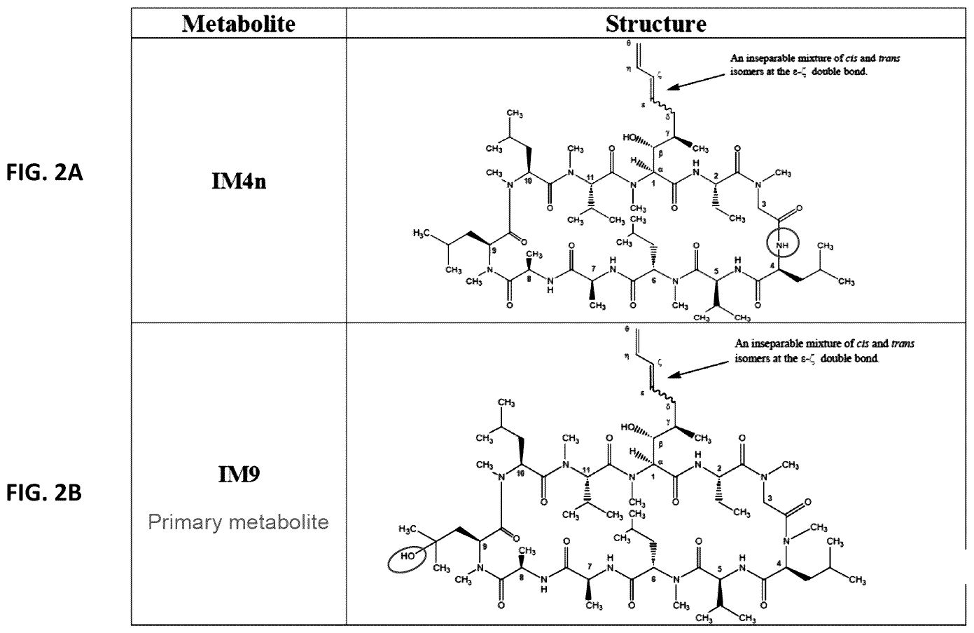

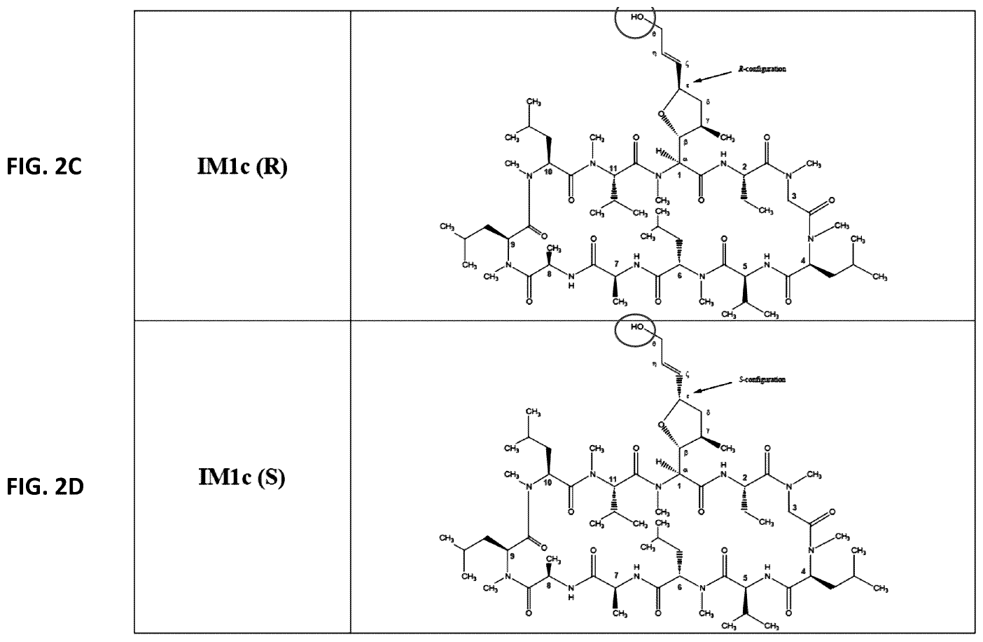

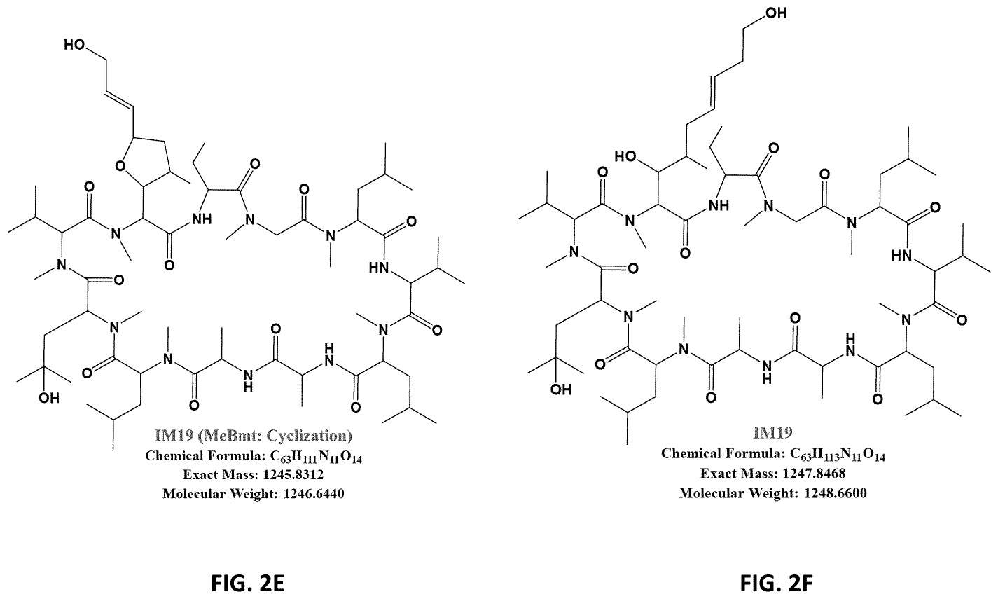

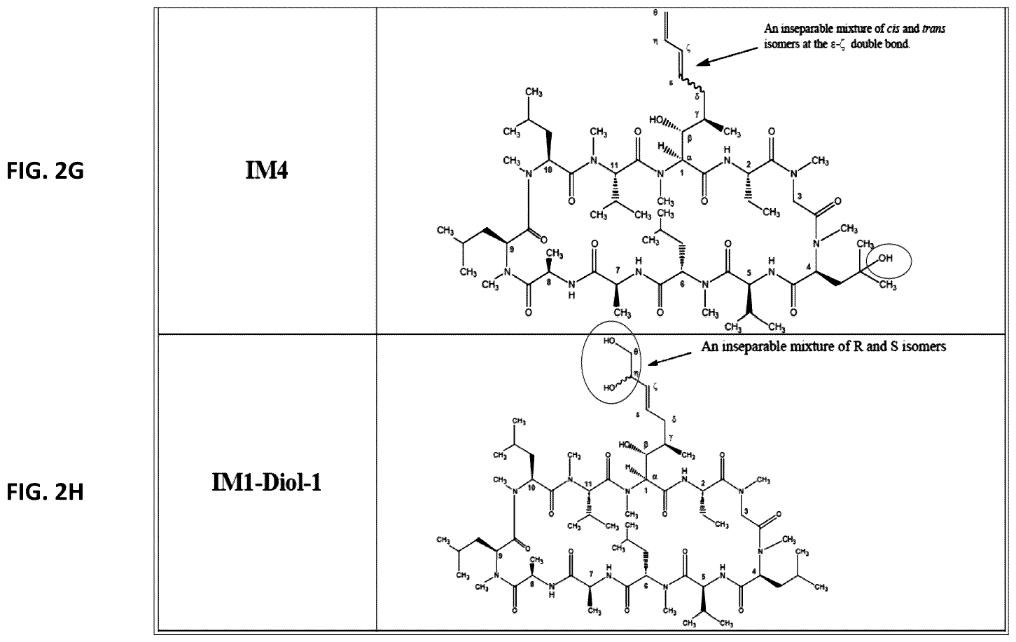

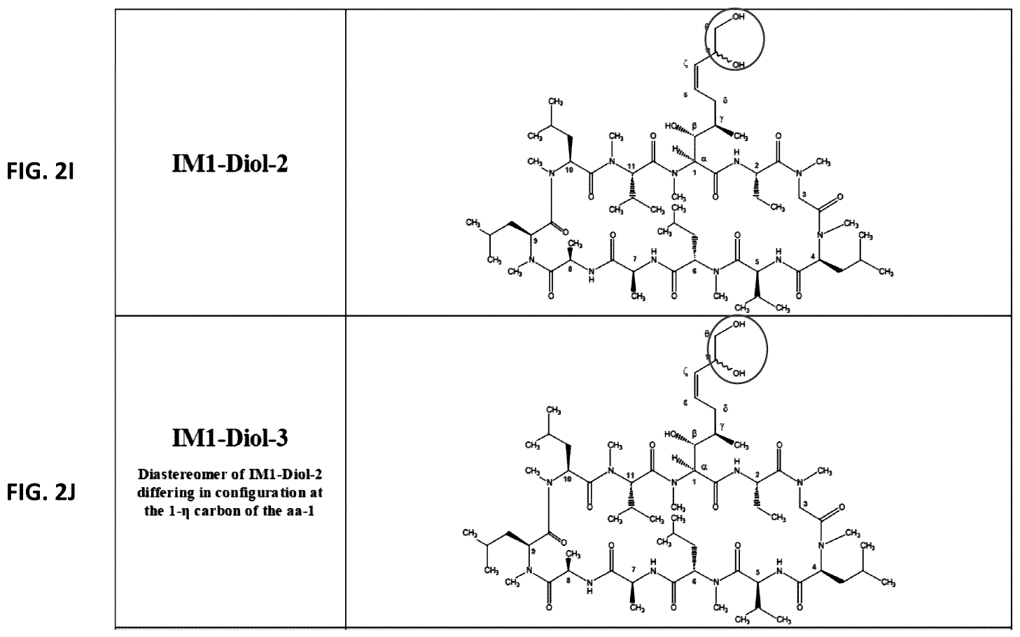

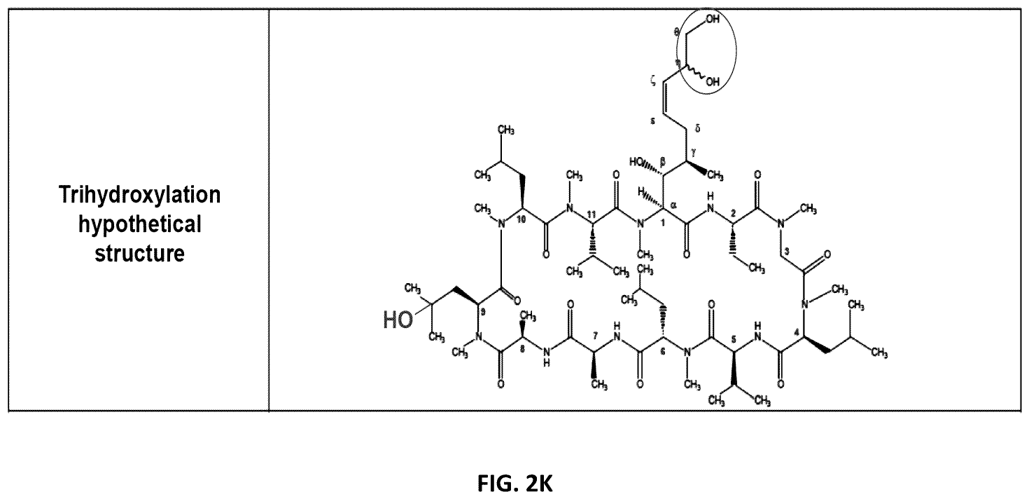

Major metabolites of voclosporin include IM4n, IM9, IM1c(R), IM1c(S), IM19 (MeBmt: Cyclization), IM19, IM4, IMI-Diol-1, IMI-Diol-2, and IMI-Diol-3, and a trihydroxylated predicted voclosporin metabolite (see FIG. 2A-FIG. 2K describing structures of exemplary voclosporin metabolites).

Mice were administered either cyclosporine A or voclosporin via tail vein injection, and kidneys were harvested after administration. Kidneys were cryosectioned and covered with a matrix for MALDI-MSI analysis. The sample was measured in a raster process in the mass spectrometer, resulting in a spatially resolved mass spectra. The detected molecules were visualized as a color intensity map (m/z 0%-100%). Histological staining of the sample was then performed. Histological staining and the MALDI-MSI measurements of the same sample were co-registered.

Table 7 below shows the actual and experimental mass to charge ratio (m/z) along with the mass accuracy (ppm) of cyclosporine A and voclosporin metabolites.

TABLE 7
Cyclosporine A and Voclosporin metabolites
in mouse kidney after IV administration

| | Actual | Experimental | Mass |
|---|---|---|---|
| | m/z | m/z | Accuracy |
| Species | ([M + Na]+) | ([M + Na]+) | (ppm) |
| [CsA + Na]⁺ | 1224.8311 | 1224.8329 | 1.47 |
| [VSP + Na]⁺ | 1236.8311 | 1236.8327 | 1.29 |
| [AM4n + Na]⁺ | 1210.8155 | 1210.8251 | 7.84 |
| [IM4n + Na]⁺ | 1222.8155 | 1222.8136 | 1.55 |
| [AM1/AM1c/AM9 + Na]⁺ | 1240.8261 | 1240.8224 | 2.98 |
| [IM1c/IM4/IM9 + Na]⁺ | 1252.8261 | 1252.8282 | 1.68 |
| [AM1c9/AM19 + Na]⁺ | 1256.8210 | 1256.8176 | 2.71 |
| [IM19 (MeBmt: | 1268.8210 | 1268.8185 | 1.97 |
| Cyclization) + Na]⁺ | | | |
| [IM1-Diol-1/IM19 + Na]⁺ | 1270.8366 | 1270.8136 | 18.1 |
| [(IM19 + OH) + Na]⁺ | 1286.8315 | 1286.8370 | 4.27 |
| Trihydroxylation | | | |
| (hypothetical) | | | |

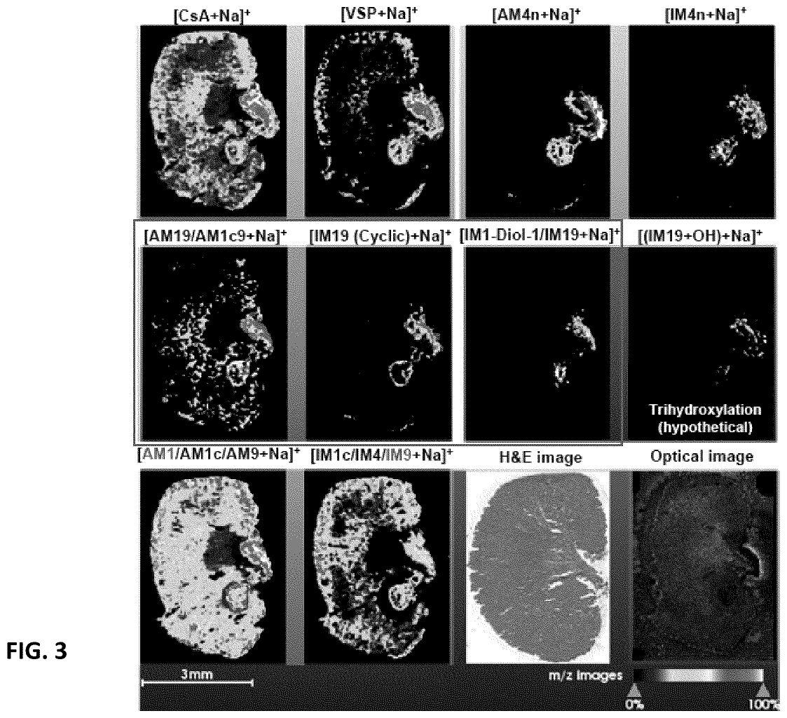

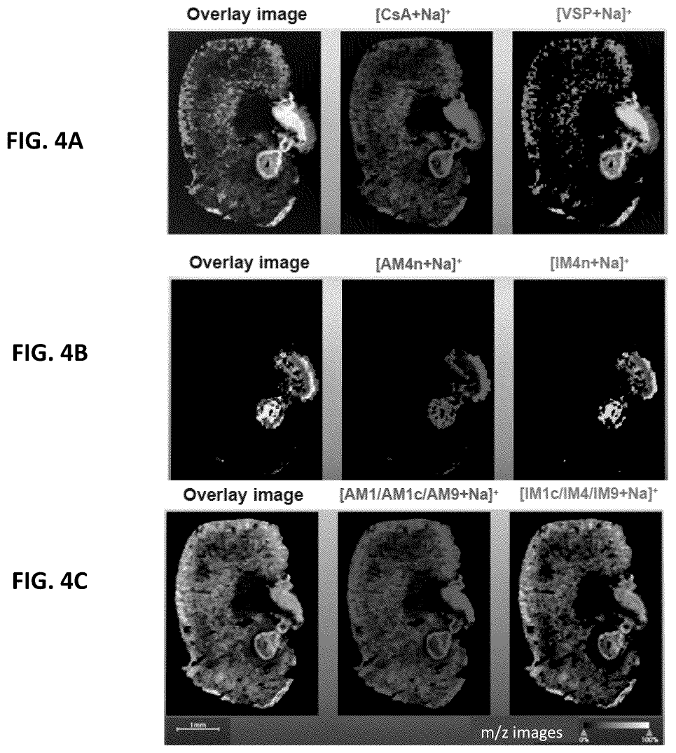

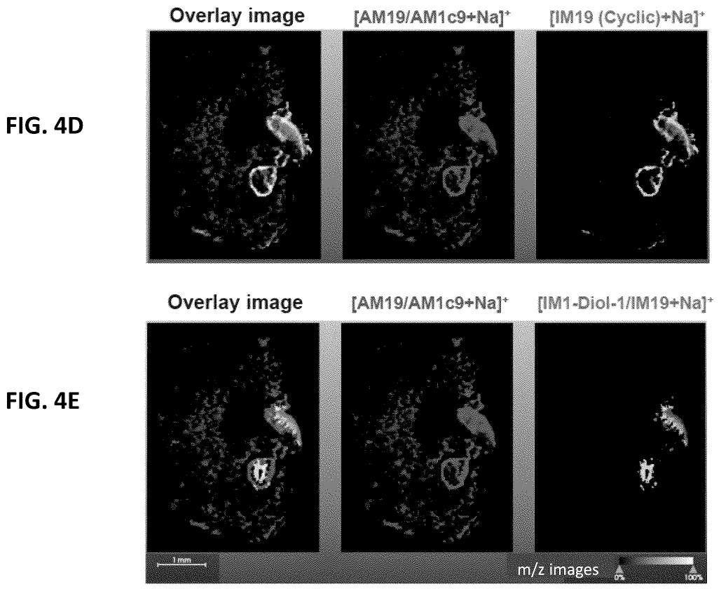

FIG. 3 shows MALDI-MSI, optical imaging, and hematoxylin and eosin (H&E) staining of exemplary mouse kidneys. Compared to cyclosporine A, the distribution and the intensity of voclosporin were relatively weak throughout the kidney, particularly in the cortex ([VSP+Na]⁺ vs [CsA+Na]⁺). Reduced kidney accumulation, distribution and intensity were evident for some voclosporin metabolites when compared to cyclosporine A metabolites. Similar to other voclosporin metabolites, the hypothetical trihydroxylated metabolite [(IM19+OH)]⁺ Nar appeared not to accumulate substantially in the kidney. In contrast to other voclosporin metabolites and the parent compound itself, [IM1c/IM4/IM9+Na]⁺ was distributed in the kidney to a greater extent and displayed a greater overall intensity. FIG. 4A-FIG. 4E show a series of MALDI-MSI images of overlaid cyclosporine A and voclosporin in addition to the molecular localization of each compound or metabolite thereof separately. Voclosporin and its metabolites, with the exception of [IM1c/IM4/IM9+Na]⁺, appeared to be distributed in the kidney to a substantially lower extent than cyclosporine A and its metabolites, and with a substantially lower intensity.

Together, these results are consistent with a distinct kidney distribution of voclosporin and its metabolites relative to cyclosporine A and its metabolites. The prevalent distribution and intensity of cyclosporine A and its metabolites in the kidney may partly explain the compound's nephrotoxicity. In contrast, the reduced intensity, accumulation and distribution of voclosporin and its metabolites throughout the kidney support voclosporin's enhanced tolerability and reduced nephrotoxicity, and support the use of voclosporin therapy in subjects in need of CNI therapy, particularly for those who may be susceptible to or may exhibit increased risk or susceptibility to CNI nephrotoxicity.

### Example 21: Assessment of Lipid Profiles in Lupus Nephritis Patients Treated with Voclosporin

Dyslipidemia is a potential side effect of immunosuppressants used to treat lupus nephritis (LN). LN is associated with an increased risk of cardiovascular disease, which can be exacerbated by dyslipidemia associated with inflammation and the immunosuppressant. Voclosporin is structurally similar to cyclosporine A, for which dyslipidemia is a known side effect. Voclosporin treatment for other disease states has been reported to have a neutral impact on mean lipid levels. This Example assesses the effect of voclosporin on the lipid levels of subjects with LN. The effect of voclosporin in combination with immunosuppressive therapy on lipid levels of subjects with LN was investigated.

Subjects with biopsy-proven active lupus nephritis (class III, IV, or V±III/IV) and proteinuria ≥1.5 mg/mg (≥2 mg/mg for class V) were enrolled in Phase 2 and Phase 3 clinical studies to assess the efficacy and safety of voclosporin in comparison to a placebo (control). All patients were concurrently administered mycophenolate mofetil (MMF) (target 1 g BID) and rapidly-tapered, low-dose steroids. Pooled data from the similarly designed clinical studies demonstrated that the addition of voclosporin to MMF and low-dose steroids resulted in significantly higher complete renal response rates (CRR) at one year of treatment (43.7% vs. 23.3%; OR 2.76; P<0.0001).

The pooled dataset was further analyzed to evaluate the impact of voclosporin on lipid levels in subjects with LN. Pooled data included 268 patients in the voclosporin (23.7 mg BID) arm and 266 patients in the placebo-treated control arm. Fasting total cholesterol, low-density lipoprotein cholesterol (LDL), high-density lipoprotein cholesterol (HDL), and triglycerides were measured at baseline and at a one year follow-up in a central, CLIA-certified laboratory.

Table 8 shows changes in the lipid profiles of subjects treated with voclosporin or placebo (control) from baseline to one year. Mean values for total cholesterol, LDL, and triglycerides were above the respective normal ranges at baseline and decreased during the study in both voclosporin and placebo arms. At one year, the decreases from baseline were significantly greater in the voclosporin arm than the control arm for total cholesterol (P=0.0062) and LDL (P=0.023). The overall reduction from baseline in triglycerides was also greater in voclosporin-treated patients at one year (P=0.0768). At one year, the percentage of patients with total cholesterol in the normal range increased by about 42% (from 17.3% at baseline to 59.2%) in the voclosporin arm versus about a 29% increase (from 19.4% at baseline to 48.3%) in the control arm. The percentage with normal LDL increased by about 42% (from 29.7% at baseline to 71.4%) in the voclosporin arm compared to an increase of about 21% (from 34.5% at baseline to 55.2%) in the control arm. The percentage of subjects with normal triglycerides increased from 32.3% to 56.1% in the voclosporin arm and from 36.1% to 53.6% in the control arm. Mean HDL levels were within the normal range at baseline in both arms and remained stable throughout the study.

TABLE 8
Lipid Profile at Baseline and One Year

| | |
|---|---|
| Control | Voclosporin |
| N = 266 | N = 268 |

| | |
|---|---|
| Change from | Change from |
| Baseline to | Baseline to |

| | | | |
|---|---|---|---|
| Observed | One Year | Observed | One Year |

Total cholesterol, mg/dL (normal range: 100 to 200)

| Baseline | n | 263 | | | | 266 | | | |
|---|---|---|---|---|---|---|---|---|---|
| | Mean (SD) | 273.7 | (86.63) | | | 288.8 | (111.46) | | |
| One Year | n | 180 | | 177 | | 196 | | 196 | |
| | Mean (SD) | 209.5 | (59.97) | −61.6 | (92.18) | 196.6 | (53.85) | −82.6 | (93.06) |
| | LS Mean Difference | | | | | | | −15.1 | |
| | Voclosporin vs | | | | | | | (−25.9, −4.3) | |
| | Control (95% CI) | | | | | | | 0.0062 | |
| | p-value | | | | | | | | |

Triglycerides, mg/dL (normal range: 50 to 150)

| Baseline | n | 266 | | | | 267 | | | |
|---|---|---|---|---|---|---|---|---|---|
| | Mean (SD) | 214.5 | (123.01) | | | 239.6 | (148.43) | | |
| One Year | n | 183 | | 183 | | 198 | | 198 | |
| | Mean (SD) | 157.5 | (103.28) | −48.4 | (117.08) | 148.4 | (96.70) | −74.1 | (110.62) |
| | LS Mean Difference | | | | | | | −15.9 | |
| | Voclosporin vs | | | | | | | (-33.6, 1.7) | |
| | Control (95% CI) | | | | | | | 0.0768 | |
| | p-value | | | | | | | | |

LDL cholesterol, mg/dL (normal range: 50 to 130)

| Baseline | n | 246 | | | | 240 | | | |
|---|---|---|---|---|---|---|---|---|---|
| | Mean (SD) | 168.6 | (72.52) | | | 174.0 | (84.98) | | |
| One Year | n | 174 | | 165 | | 193 | | 182 | |
| | Mean (SD) | 124.6 | (47.85) | −44.7 | (77.87) | 114.3 | (41.75) | −57.4 | (78.51) |
| | LS Mean Difference | | | | | | | −10.3 | |
| | Voclosporin vs | | | | | | | (−19.2, −1.4) | |
| | Control (95% CI) | | | | | | | 0.023 | |
| | p-value | | | | | | | | |

HDL cholesterol, mg/dL (normal range: 35 to 60)

| Baseline | n | 263 | | | | 266 | | | |
|---|---|---|---|---|---|---|---|---|---|
| | Mean (SD) | 58.6 | (23.27) | | | 57.2 | (21.33) | | |
| One Year | n | 180 | | 177 | | 196 | | 196 | |
| | Mean (SD) | 52.9 | (17.15) | −6.1 | (17.12) | 51.9 | (17.28) | −5.9 | (17.49) |
| | LS Mean Difference | | | | | | | −0.6 | |
| | Voclosporin vs | | | | | | | (−3.4, 2.2) | |
| | Control (95% CI) | | | | | | | 0.6708 | |
| | p-value | | | | | | | | |

From baseline to the one year follow-up, total cholesterol, LDL cholesterol, and triglyceride levels improved in both treatment arms. Along with higher CRR rates, the voclosporin arm exhibited greater reductions in lipids and an overall greater percentage of subject entry to normal lipid ranges. Given the risk of cardiovascular disease in LN patients, the improvements in CRR and favorable impact on lipid levels observed in the clinical studies further support the use of the CNI voclosporin for the treatment of LN and other diseases.

### Example 22: Long-Term Safety and Tolerability Continuation Study

Subjects who previously completed twelve months of voclosporin treatment in combination with mycophenolate mofetil (MMF) and steroids for lupus nephritis (LN), were enrolled in a long-term continuation study for an additional two years with the same treatment.

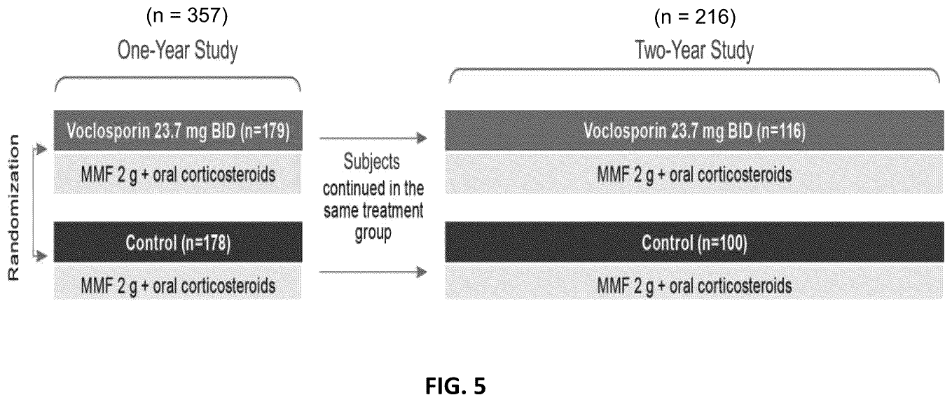

The continuation study evaluated voclosporin compared to placebo, in combination with mycophenolate mofetil (MMF) and low-dose steroids, in subjects with lupus nephritis, generally as described in Example 21. The continuation study involved 216 subjects out of the 357 subjects who were included in the initial 1 year study, and continued in the same treatment groups: 116 patients in the voclosporin group, receiving the same treatment of voclosporin at 23.7 mg twice daily, in combination with MMF at 1 g twice daily with low-dose steroids, and 100 patients in the control group receiving placebo in combination with MMF at 1 g twice daily with low-dose steroids, generally as shown in FIG. 5, for an additional 24 months. subjects in the voclosporin group and 78 subjects in the control group received 36 months of total treatment at the completion of the study Subject demographics are shown in Table 9.

TABLE 9
Demographics

| | |
|---|---|
| Placebo | Voclosporin |
| n = %(n = 100) | m = % (n = 116) |

Age, years

| | | | | |
|---|---|---|---|---|
| Mean (SD) | 35.4 | (11.64) | 32.3 | (10.31) |

Sex, n (%)

| | | | | |
|---|---|---|---|---|
| Female | 88.0 | (88) | 90.5 | (105) |
| Male | 12.0 | (12) | 9.5 | (11) |

Race, n (%)

| | | | | |
|---|---|---|---|---|
| White | 40.0 | (40) | 37.9 | (44) |
| Asian | 30.0 | (30) | 25.9 | (30) |
| Black | 7.0 | (7) | 15.5 | (18) |
| Other | 13.0 | (13) | 6.9 | (8) |

Biopsy Class, n (%)

| | | | | |
|---|---|---|---|---|
| Pure Class III or IV | 58.0 | (58) | 67.2 | (78) |
| Pure Class V | 14.0 | (14) | 14.7 | (17) |
| Mixed Class V | 28.0 | (28) | 18.1 | (21) |

Region, n (%)

| | | | | |
|---|---|---|---|---|
| North and Latin America | 36.0 | (36) | 42.2 | (49) |
| Europe and South Africa | 37.0 | (37) | 32.8 | (38) |
| Asia | 27.0 | (27) | 25.0 | (29) |

Table 10 shows observed values in the mean corrected estimated glomerular filtration rate (eGFR) and mean urine protein creatinine ratio (UPCR) at pre-treatment baseline and at one year of treatment (Month 12) baseline.

TABLE 10
Corrected eGFR and UPCR Values at Pre-
Treatment and at One Year of Treatment

| | |
|---|---|
| Placebo | Voclosporin |
| N = 100 | N = 116 |

Corrected eGFR, mL/min/1.73 m², mean (SD)

| Baseline | n | 100 | | 116 | |
|---|---|---|---|---|---|
| | Mean (SD) | 78.7 | (16.58) | 79.0 | (15.05) |
| Month 12 | n | 100 | | 116 | |
| | Mean (SD) | 83.3 | (12.61) | 80.7 | (13.53) |

UPCR, mg/mg, mean (SD)

| Baseline | n | 100 | | 116 | |
|---|---|---|---|---|---|
| | Mean (SD) | 3.868 | (2.4764) | 3.941 | (2.5766) |
| Month 12 | n | 100 | | 116 | |
| | Mean (SD) | 1.47 | (1.640) | 0.86 | (1.363) |

Oral Steroid Dose

| | | | | | |
|---|---|---|---|---|---|
| Month 12 | Mean (SD), | 4.1104 | (3.395) | 3.3456 | (2.942) |
| | mg/day | | | | |
| | ≤2.5 mg/day, | 85.0 | (85) | 87.9 | (102) |
| | n (%) | | | | |

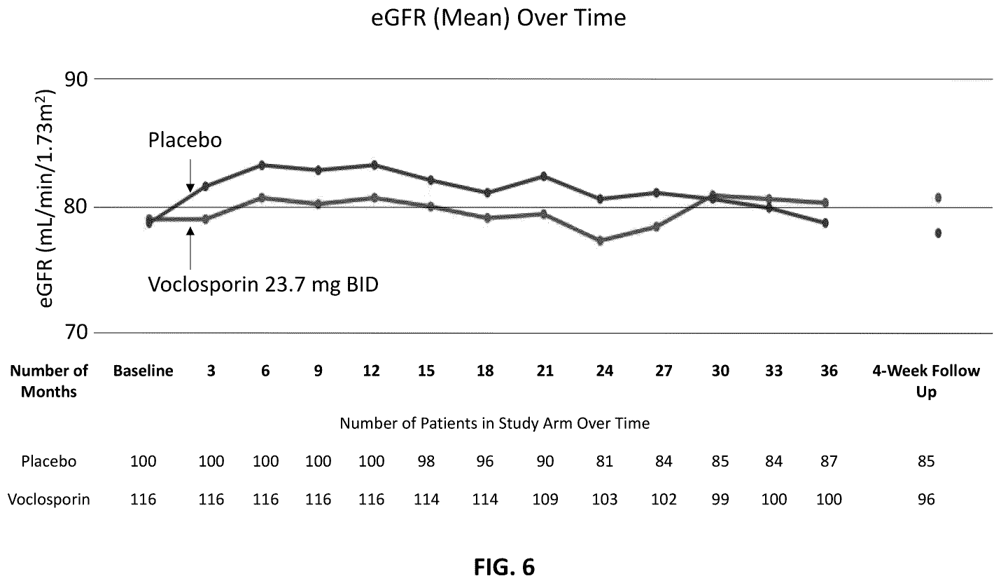

In the 116 subjects in the voclosporin-treated group, mean estimated glomerular filtration rate (eGFR) was stable over 36 months as shown in FIG. 6. Additionally, compared to the active control group, the voclosporin-treated group showed an increase from baseline eGFR of +2.7 mL/min, at 36 months. Mean eGFR was stable over 36 months in the voclosporin-treated group.

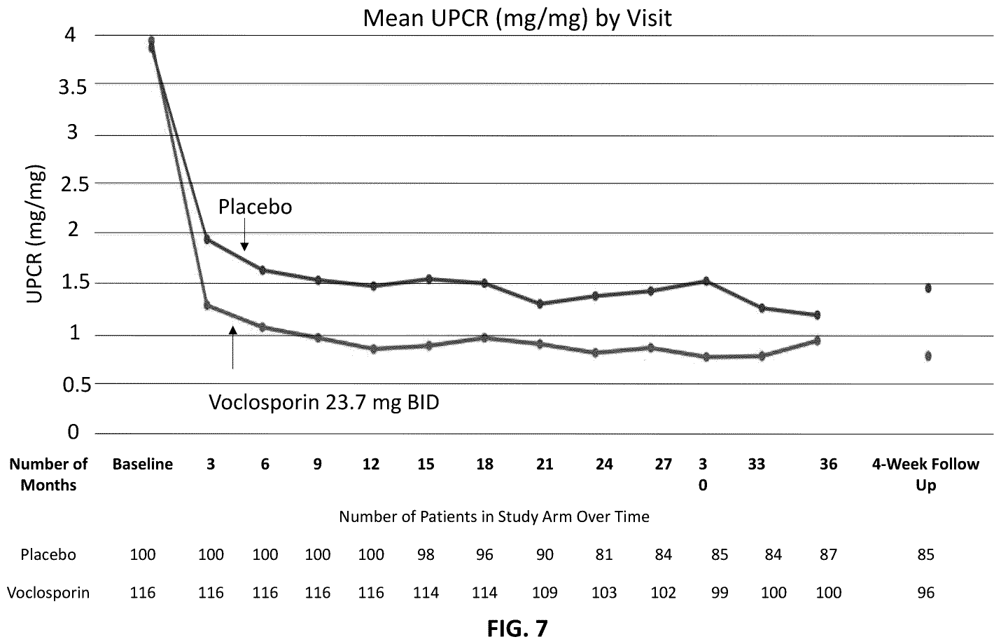

The mean UPCR was lower in the voclosporin-treated groups at all time points during the three years as shown in FIG. 7.

Voclosporin was well tolerated with no unexpected safety signals observed. Table 11 shows a summary of adverse events. There were comparable serious adverse event rates in both study arms, 19% for the voclosporin-treated group and 24% for the active control group. Additionally, there were no deaths in the voclosporin-treated group whereas there were four deaths in the active control group. The active control group had a higher percentage of withdrawals (15.0% withdrawal) compared to the voclosporin-treated group (12.9% withdrawal).

TABLE 11
Summary of Adverse Events

| | Placebo | | Voclosporin | |
|---|---|---|---|---|
| | (n = 100) | | (n = 116) | |
| | % (n) | | % (n) | |
| Any Adverse Event | 83.0 | (83) | 88.8 | (103) |
| Treatment-Related Adverse Event | 25.0 | (25) | 27.6 | (32) |
| Serious Adverse Event | 24.0 | (24) | 19.0 | (22) |
| Treatment-Related Serious Adverse Event | 2.0 | (2) | 0.9 | (1) |
| Adverse Event Leading to Voclosporin/ | 18.0 | (18) | 10.3 | (12) |
| Placebo Discontinuation | | | | |
| Deaths | 4.0 | (4) | 0 | |
| Disease-Related Adverse Event | 43.0 | (43) | 51.7 | (60) |
| Disease-Related Serious Adverse Event | 13.0 | (13) | 6.9 | (8) |

The results of the long-term continuation study of three years, evaluating the long-term safety and tolerability of voclosporin for the treatment of adults with active lupus nephritis (LN), a serious complication in subjects with systemic lupus erythematosus (SLE), in combination with background immunosuppressive therapy, support a long-term treatment with voclosporin for subjects with LN. The results show long-term safety and tolerability, minimal impact on eGFR even after up to three years of treatment, while maintaining effective treatment and substantial reduction in proteinuria

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method to reduce chronic calcineurin inhibitor nephrotoxicity in treatment of lupus nephritis, which method comprises administering to a subject diagnosed with lupus nephritis, a predetermined daily dosage of 23.7 mg BID of voclosporin over a projected treatment period of at least 100 weeks, said method further comprising:
(a) assessing the estimated Glomerular Filtration Rate (eGFR) of said subject at at least a first time point and a second time point on different days of said treatment period; and
(b) (i) if the eGFR of said subject decreases by more than 20-45% to below a predetermined value of 60 ml/min/1.73 m², between said first and second time points, reducing the daily dosage by increment(s) of 7.9 mg BID;
(ii) if the eGFR of said subject decreases by less than 20-45%, between said first and second time points, continuing administering the same predetermined daily dosage of voclosporin to said subject.

2. The method of claim 1, wherein the first time point is immediately preceding initiating said administration of voclosporin.

3. The method of claim 1, further comprising identifying said subject as appropriate for said method prior to conducting said method on said subject by:
(a) determining that the urine protein creatinine ratio (UPCR) of said subject is >1 mg/mg as measured by first morning void or 24 hour urine; and
(b) determining said subject has an eGFR as measured by Chronic Kidney Disease Epidemiology Collaboration equation (CKD-EP1) of >45 ml/min/1.73 m²,
wherein if the conditions of (a) and (b) are met, said subject is identified as appropriate for said method.

4. The method of claim 1, wherein said subject has increased susceptibility to chronic calcineurin inhibitor nephrotoxicity.

5. The method of claim 1, wherein said subject exhibits one or more of:
(a) variability in P-glycoprotein expression and/or activity;
(b) variability in CYP3A4/5 expression and/or activity;
(c) older kidney age;
(d) salt depletion;
(e) the use of nonsteroidal anti-inflammatory drugs;
(f) genetic polymorphisms in TGF-β and/or ACE; and/or
(g) glomerulonephropathy.

6. The method of claim 1, further comprising:
(a) assessing the interstitial fibrosis and tubular atrophy of said subject by renal biopsies at at least a first time point and a second time point on different days of said treatment period; and
(b) (i) if interstitial fibrosis and tubular atrophy is observed in >5% in cortical area, between said first and second time points, reducing the daily dosage by increment(s) of 7.9 mg BID or stopping the administering of voclosporin to said subject;
(ii) if interstitial fibrosis and tubular atrophy is observed in <5% in cortical area, between said first and second time points, continuing administering the same predetermined daily dosage of voclosporin to said subject.

7. The method of claim 1, further comprising:
(a) assessing the presence of medial arteriolar hyalinosis of said subject by renal biopsies at at least a first time point and a second time point on different days of said treatment period; and
(b) (i) if medial arteriolar hyalinosis is present, between said first and second time points, reducing the daily dosage by increment(s) of 7.9 mg BID or stopping the administering of voclosporin to said subject;
(ii) if medial arteriolar hyalinosis is not present, between said first and second time points, continuing administering the same predetermined daily dosage of voclosporin to said subject.

8. The method of claim 1, further comprising:
(a) assessing the presence of glomerular injury of said subject by renal biopsies at at least a first time point and a second time point on different days of said treatment period, and
(b) (i) if glomerular injury is present, between said first and second time points, reducing the daily dosage by increment(s) of 7.9 mg BID or stopping the administering of voclosporin to said subject;
(ii) if glomerular injury is not present, between said first and second time points, continuing administering the same predetermined daily dosage of voclosporin to said subject.

9. The method of claim 1, further comprising:
(a) assessing the presence of juxtaglomerular apparatus (JGA) hyperplasia of said subject by renal biopsies at at least a first time point and a second time point on different days of said treatment period, and
(b) (i) if JGA hyperplasia is present, between said first and second time points, reducing the daily dosage by increment(s) of 7.9 mg BID or stopping the administering of voclosporin to said subject;
(ii) if JGA hyperplasia is not present, between said first and second time points, continuing administering the same predetermined daily dosage of voclosporin to said subject.

10. The method of claim 1, further comprising:
(a) assessing the presence of tubular microcalcifications of said subject by renal biopsies at at least a first time point and a second time point on different days of said treatment period, and
(b) (i) if tubular microcalcifications are present, between said first and second time points, reducing the daily dosage by increment(s) of 7.9 mg BID or stopping the administering of voclosporin to said subject;
(ii) if tubular microcalcifications are not present, between said first and second time points, continuing administering the same predetermined daily dosage of voclosporin to said subject.

11. The method of claim 1, further comprising:
(a) assessing the P-glycoprotein (P-gp) expression of said subject by renal biopsies at at least a first time point and a second time point on different days of said treatment period, and
(b) (i) if loss of expression of P-gp is more than a predetermined value, between said first and second time points, reducing the daily dosage by increment(s) of 7.9 mg BID or stopping the administering of voclosporin to said subject;
(ii) if loss of expression of P-gp is less than the predetermined value, between said first and second time points, continuing administering the same predetermined daily dosage of voclosporin to said subject,
wherein the predetermined value for loss of expression of P-gp is 10% loss of P-gp expression in tubules in the cortical area.

12. The method of claim 1, further comprising:
(a) assessing the Calcineurin Inhibitor (CNI) Nephrotoxicity and/or Banff Scores of said subject by renal biopsies at at least a first time point and a second time point on different days of said treatment period, and
(b) (i) if the CNI Nephrotoxicity and/or Banff Scores are outside of predetermined ranges, between said first and second time points, reducing the daily dosage by increment(s) of 7.9 mg BID or stopping the administering of voclosporin to said subject;
(ii) if the CNI Nephrotoxicity and/or Banff Scores are within predetermined ranges, between said first and second time points, continuing administering the same predetermined daily dosage of voclosporin to said subject,
wherein:
(a) the predetermined range of CNI Nephrotoxicity Score is 0-3; and
(b) the predetermined range of Banff Score is 0-3.

13. The method of claim 1, further comprising:
(a) assessing one or more of the National Institutes of Health Activity Index (NIH-AI), the National Institutes of Health Chronicity Index (NIH-CI), and the Tubulointerstitial Activity Index (TIAI) of said subject by renal biopsies at at least a first time point and a second time point on different days of said treatment period, and
(b) (i) if the NIH-AI, NIH-CI, and/or TIAI are outside of predetermined ranges, between said first and second time points, reducing the daily dosage by increment(s) of 7.9 mg BID or stopping the administering of voclosporin to said subject;
(ii) if the NIH-AI, NIH-CI, and/or TIAI are within predetermined ranges, between said first and second time points, continuing administering the same predetermined daily dosage of voclosporin to said subject,
wherein:
(a) the predetermined range of NIH-AI is 0-6;
(b) the predetermined range of NIH-CI is 0-3; and
(c) the predetermined range of TIAI is 0-5.

14. The method of claim 1, further comprising:
(a) assessing urine anion gap (UAG) in urinary specimens of said subject at at least a first time point and a second time point on different days of said treatment period, and
(b) (i) if the UAG is outside of a predetermined range, between said first and second time points, reducing the daily dosage by increment(s) of 7.9 mg BID or stopping the administering of voclosporin to said subject;
(ii) if the UAG is within the predetermined range, between said first and second time points, continuing administering the same predetermined daily dosage of voclosporin to said subject,
wherein the predetermined range of UAG is between 20-90 mEq/L.

15. The method of claim 1, further comprising:
(a) assessing hyperkalemia, hypomagnesemia, magnesium wasting and/or hyperuricemia in serum and urine samples of said subject at at least a first time point and a second time point on different days of said treatment period, and
(b) (i) if hyperkalemia, hypomagnesemia, magnesium wasting and/or hyperuricemia are detected, between said first and second time points, reducing the daily dosage by increment(s) of 7.9 mg BID or stopping the administering of voclosporin to said subject;
(ii) if hyperkalemia, hypomagnesemia, magnesium wasting and hyperuricemia are not detected, between said first and second time points, continuing administering the same predetermined daily dosage of voclosporin to said subject,
wherein:
(a) hyperkalemia is determined by a serum potassium level of >5 mmol/L;
(b) hypomagnesemia is determined by a serum magnesium level less than 1.4 mg/dL;
(c) magnesium wasting is determined by a urine magnesium level of more than 2 mEq; and
(d) hyperuricemia is determined by a serum uric acid level of >7.0 mg/dL.

16. The method of claim 1, further comprising:
(a) assessing serum creatinine (SCr) and/or serum cystatin C (SCysC) in serum samples of said subject at at least a first time point and a second time point on different days of said treatment period, and
(b) (i) if the SCr and/or ScysC levels are elevated above predetermined ranges, between said first and second time points, reducing the daily dosage by increment(s) of 7.9 mg BID or stopping the administering of voclosporin to said subject;
(ii) if the SCr and/or ScysC levels are within predetermined ranges, between said first and second time points, continuing administering the same predetermined daily dosage of voclosporin to said subject,
wherein:
(a) the predetermined range of SCr level is 0.84-1.21 mg/dL; and
(b) the predetermined range of SCysC level is below 1 mg/L.

17. The method of claim 1, further comprising:
(a) assessing creatinine clearance (CrCl) and/or blood urea nitrogen (BUN) in serum samples of said subject at at least a first time point and a second time point on different days of said treatment period, and
(b) (i) if the CrCl level is decreased below a first predetermined range and/or if BUN level is elevated above a second predetermined range, between said first and second time points, reducing the daily dosage by increment(s) of 7.9 mg BID or stopping the administering of voclosporin to said subject;
(ii) if the CrCl and/or BUN levels are within predetermined ranges, between said first and second time points, continuing administering the same predetermined daily dosage of voclosporin to said subject,
wherein:
(a) the first predetermined range of CrCl level is 137-150 mL/min in males and 128-130 mL/min in females; and
(b) the second predetermined range of BUN level is 7 to 20 mg/dL.

18. The method of claim 1, further comprising:
(a) assessing renal vascular resistance and/or renal plasma flow (RPF) of said subject at at least a first time point and a second time point on different days of said treatment period, and
(b) (i) if the renal vascular resistance and/or RPF are altered outside of predetermined values, between said first and second time points, reducing the daily dosage by increment(s) of 7.9 mg BID or stopping the administering of voclosporin to said subject;
(ii) if the renal vascular resistance and/or RPF remain within predetermined values, between said first and second time points, continuing administering the same predetermined daily dosage of voclosporin to said subject,
wherein the predetermined value of RPF is 600 mL/min.

19. The method of claim 1, further comprising:
(a) assessing albuminuria in morning/random urinary specimens of said subject at at least a first time point and a second time point on different days of said treatment period, and
(b) (i) if the albuminuria is detected, between said first and second time points, reducing the daily dosage by increment(s) of 7.9 mg BID or stopping the administering of voclosporin to said subject;
(ii) if the albuminuria is not detected, between said first and second time points, continuing administering the same predetermined daily dosage of voclosporin to said subject,
wherein albuminuria is determined by the presence of albumin/creatinine ratios of >30 mg/g.

20. The method of claim 1, wherein the method does not result in a substantial increase or decrease of the level of one or more urinary electrolytes between said first and second time points.

21. The method of claim 1, further comprising:
(a) assessing the level of one or more urinary electrolytes of said subject at at least a first time point and a second time point on different days of said treatment period, and
(b) (i) if the level of the one or more urinary electrolyte is decreased or increased by more than a predetermined value, between said first and second time points, reducing the daily dosage by increment(s) of 7.9 mg BID or stopping the administering of voclosporin to said subject;
(ii) if the level of the one or more urinary electrolyte is decreased or increased by less than the predetermined value, between said first and second time points, continuing administering the same predetermined daily dosage of voclosporin to said subject,
wherein the urinary electrolyte is selected from one or more of magnesium, sodium and potassium,
wherein the predetermined value of magnesium is about 20 mg/dL,
wherein the predetermined value of sodium is about 50 mmol/L, and
wherein the predetermined value of potassium is about 10 mmol/L.

22. The method of claim 1, wherein the method does not result in a substantial dyslipidemia at said second time point.

23. The method of claim 1, further comprising:
(a) assessing the level of one or more lipids of said subject at at least a first time point and a second time point on different days of said treatment period, and
(b) (i) if the level of the one or more lipid is outside of a predetermined range, at said second time point, reducing the daily dosage by increment(s) of 7.9 mg BID or stopping the administering of voclosporin to said subject;
(ii) if the level of the one or more lipid is within a predetermined range, at said second time point, continuing administering the same predetermined daily dosage of voclosporin to said subject,
wherein the one or more lipid is selected from one or more of total cholesterol, low density lipoprotein (LDL) cholesterol, and triglyceride,
wherein the predetermined range for total cholesterol is 100-200 mg/dL,
wherein the predetermined range is for triglycerides is 50-150 mg/dL, and
wherein the predetermined range for LDL is 50-130 mg/dL.

24. The method of claim 1, further comprising administering to said subject an effective amount of mycophenolate mofetil (MMF).

25. The method of claim 1, further comprising administering to said subject an effective amount of a corticosteroid.

27. The method of claim 1, wherein said treatment period is at least 150 weeks.

28. The method of claim 1, wherein said second time point is about 50-60 weeks subsequent to initiating said administration of voclosporin.
